SEQUENCE LISTING (76 sequences)

SEQ ID NO: 1 - DNA, 37 bp, Artificial Sequence
  Description of Artificial Sequence Synthetic probe
tgtgggcgaagcgggaacctcctcctctcccttcagc

SEQ ID NO: 2 - DNA, 37 bp, Artificial Sequence
  Description of Artificial Sequence Synthetic probe
tcggagcgatcacgtggcaccatggctgagacaagaa

SEQ ID NO: 3 - DNA, 35 bp, Artificial Sequence
  Description of Artificial Sequence Synthetic probe
tacgcgacgcacctgctccaacacccggcccacgg

SEQ ID NO: 4 - DNA, 38 bp, Artificial Sequence
  Description of Artificial Sequence Synthetic probe
tcctccctcacgcgcctgcagaggcacaagtggatggt

SEQ ID NO: 5 - DNA, 37 bp, Artificial Sequence
  Description of Artificial Sequence Synthetic probe
tgtggactgagcgcggatggcccaggtctgggtggtt

SEQ ID NO: 6 - DNA, 37 bp, Artificial Sequence
  Description of Artificial Sequence Synthetic probe
tcgaggcagacgcgtcccaccagctgctctcagtcaa

SEQ ID NO: 7 - DNA, 36 bp, Artificial Sequence
  Description of Artificial Sequence Synthetic probe
ttggccgagactgcaggagcgcccggtcaccagcaa

SEQ ID NO: 8 - DNA, 37 bp, Artificial Sequence
  Description of Artificial Sequence Synthetic probe
tagcggacgactgcggacgagaaactggcagcactct

SEQ ID NO: 9 - DNA, 36 bp, Artificial Sequence
  Description of Artificial Sequence Synthetic probe
tgcctgcgaagacccaagcgacacaagttcaaaatc

SEQ ID NO: 10 - DNA, 38 bp, Artificial Sequence
  Description of Artificial Sequence Synthetic probe
tgagcagcgacgccgaggcagaccagagctacaacaat

SEQ ID NO: 11 - DNA, 35 bp, Artificial Sequence
  Description of Artificial Sequence Synthetic probe
tgcgagtcccgagggtcccaatggtcagcatttcc

SEQ ID NO: 12 - DNA, 37 bp, Artificial Sequence
  Description of Artificial Sequence Synthetic probe
tggaaagcgagcggcagccaaacacgctggtaccatt

SEQ ID NO: 13 - DNA, 25 bp, Homo sapiens
caggcatttttactgaccaagttct

SEQ ID NO: 14 - DNA, 20 bp, Homo sapiens
cccgtgcacggaaaatacag

SEQ ID NO: 15 - DNA, 18 bp, Homo sapiens
caagattggcggcattgg

SEQ ID NO: 16 - DNA, 26 bp, Homo sapiens
ttgtgcagaaaaacaagtcctaaagt

SEQ ID NO: 17 - DNA, 26 bp, Homo sapiens
cacattgatgtctcctgctgtactaa

SEQ ID NO: 18 - DNA, 26 bp, Homo sapiens
cagattacaccaaagactgtttccaa

SEQ ID NO: 19 - DNA, 28 bp, Homo sapiens
tgagttgtgactggtgtaaatacaatga

SEQ ID NO: 20 - DNA, 26 bp, Homo sapiens
gggaagatgatgacactgcattataa

SEQ ID NO: 21 - DNA, 20 bp, Homo sapiens
actgatgaccccaggagcaa

SEQ ID NO: 22 - DNA, 27 bp, Homo sapiens
ccaaagacagtcttctaattcctcatt

SEQ ID NO: 23 - DNA, 22 bp, Homo sapiens
ggtgctgaaggctgattacgtt

SEQ ID NO: 24 - DNA, 21 bp, Homo sapiens
accgctaaaggaggcgatatc

SEQ ID NO: 25 - DNA, 22 bp, Homo sapiens
actctccccttgtccactgatg

SEQ ID NO: 26 - DNA, 22 bp, Homo sapiens
cctgtgaagacctcagcaacct

SEQ ID NO: 27 - DNA, 14 bp, Homo sapiens
cccggccgcaggat

SEQ ID NO: 28 - DNA, 24 bp, Homo sapiens
tgattggacatgacacaggataca

SEQ ID NO: 29 - DNA, 18 bp, Homo sapiens
agccccactgcggtactg

SEQ ID NO: 30 - DNA, 21 bp, Homo sapiens
tgactgcaggaagctctggat

SEQ ID NO: 31 - DNA, 24 bp, Homo sapiens
aagtcccagcctctcaaactacag

SEQ ID NO: 32 - DNA, 22 bp, Homo sapiens
tgaatgctgtgtcagtgcatga

SEQ ID NO: 33 - DNA, 20 bp, Homo sapiens
ggtggggcttccgtaagtgt

SEQ ID NO: 34 - DNA, 28 bp, Homo sapiens
tcatgccagaggtcttatattttaagag

SEQ ID NO: 35 - DNA, 21 bp, Homo sapiens
caacctcctccttggaaaacc

SEQ ID NO: 36 - DNA, 19 bp, Homo sapiens
tggggcagagggactggta

SEQ ID NO: 37 - DNA, 40 bp, Artificial Sequence
  Description of Artificial Sequence Synthetic oligonucleotide
ttggcctgttcccgctcgctgaggttcccgcttcgcccac

SEQ ID NO: 38 - DNA, 40 bp, Artificial Sequence
  Description of Artificial Sequence Synthetic oligonucleotide
cctgcgctgcgtggtgttccggtgccacgtgatcgctccg

SEQ ID NO: 39 - DNA, 40 bp, Artificial Sequence
  Description of Artificial Sequence Synthetic oligonucleotide
tcgcggacggtgagcactgcttggagcaggtgcgtcgcgt

SEQ ID NO: 40 - DNA, 40 bp, Artificial Sequence
  Description of Artificial Sequence Synthetic oligonucleotide
ttcccgctggtgccactccgctgcaggcgcgtgagggagg

SEQ ID NO: 41 - DNA, 40 bp, Artificial Sequence
  Description of Artificial Sequence Synthetic oligonucleotide
cgctcgctttggcctgctgcgccatccgcgctcagtccac

SEQ ID NO: 42 - DNA, 40 bp, Artificial Sequence
  Description of Artificial Sequence Synthetic oligonucleotide
cgctgcgtgagggctcggacggtgggacgcgtctgcctcg

SEQ ID NO: 43 - DNA, 40 bp, Artificial Sequence
  Description of Artificial Sequence Synthetic oligonucleotide
ttccacccgcgtcgtcctgccgctcctgcagtctcggcca

SEQ ID NO: 44 - DNA, 40 bp, Artificial Sequence
  Description of Artificial Sequence Synthetic oligonucleotide
gaccgctcagcagaccgccactcgtccgcagtcgtccgct

SEQ ID NO: 45 - DNA, 40 bp, Artificial Sequence
  Description of Artificial Sequence Synthetic oligonucleotide
agcacgcttccgggacccactcgcttgggtcttcgcaggc

SEQ ID NO: 46 - DNA, 40 bp, Artificial Sequence
  Description of Artificial Sequence Synthetic oligonucleotide
aggcggtgttggctcgaggcctgcctcggcgtcgctgctc

SEQ ID NO: 47 - DNA, 40 bp, Artificial Sequence
  Description of Artificial Sequence Synthetic oligonucleotide
gcgtgtctcctgtccggccattgggaccctcgggactcgc

SEQ ID NO: 48 - DNA, 40 bp, Artificial Sequence
  Description of Artificial Sequence Synthetic oligonucleotide
ttgggctcgtgaaggcaggcttggctgccgctcgctttcc

SEQ ID NO: 49 - DNA, 22 bp, Artificial Sequence
  Description of Artificial Sequence Synthetic oligonucleotide
agcgagcgggaacaggccaatt

SEQ ID NO: 50 - DNA, 25 bp, Artificial Sequence
  Description of Artificial Sequence Synthetic oligonucleotide
ggaacaccacgcagcgcaggttttt

SEQ ID NO: 51 - DNA, 28 bp, Artificial Sequence
  Description of Artificial Sequence Synthetic oligonucleotide
gcagtgctcaccgtccgcgatttttttt

SEQ ID NO: 52 - DNA, 31 bp, Artificial Sequence
  Description of Artificial Sequence Synthetic oligonucleotide
cggagtggcaccagcgggaattttttttttt

SEQ ID NO: 53 - DNA, 34 bp, Artificial Sequence
  Description of Artificial Sequence Synthetic oligonucleotide
gcagcaggccaaagcgagcgtttttttttttttt

SEQ ID NO: 54 - DNA, 37 bp, Artificial Sequence
  Description of Artificial Sequence Synthetic oligonucleotide
gtccgagccctcacgcagcgttttttttttttttttt

SEQ ID NO: 55 - DNA, 40 bp, Artificial Sequence
  Description of Artificial Sequence Synthetic oligonucleotide
gcaggacgacgcgggtggaatttttttttttttttttttt

SEQ ID NO: 56 - DNA, 43 bp, Artificial Sequence
  Description of Artificial Sequence Synthetic oligonucleotide
tggcggtctgctgagcggtcttttttttttttttttttttttt

SEQ ID NO: 57 - DNA, 46 bp, Artificial Sequence
  Description of Artificial Sequence Synthetic oligonucleotide
gtgggtcccggaagcgtgcttttttttttttttttttttttttttt

SEQ ID NO: 58 - DNA, 49 bp, Artificial Sequence
  Description of Artificial Sequence Synthetic oligonucleotide
gcctcgagccaacaccgcctttttttttttttttttttttttttttttt

SEQ ID NO: 59 - DNA, 52 bp, Artificial Sequence
  Description of Artificial Sequence Synthetic oligonucleotide
tggccggacaggagacacgctttttttttttttttttttttttttttttttt

SEQ ID NO: 60 - DNA, 55 bp, Artificial Sequence
  Description of Artificial Sequence Synthetic oligonucleotide
gcctgccttcacgagcccaattttttttttttttttttttttttttttttttttt

SEQ ID NO: 61 - DNA, 20 bp, Artificial Sequence
  Description of Artificial Sequence Synthetic probe
gtgggcgaagcgggaacctc

SEQ ID NO: 62 - DNA, 20 bp, Artificial Sequence
  Description of Artificial Sequence Synthetic probe
cggagcgatcacgtggcacc

SEQ ID NO: 63 - DNA, 20 bp, Artificial Sequence
  Description of Artificial Sequence Synthetic probe
acgcgacgcacctgctccaa

SEQ ID NO: 64 - DNA, 20 bp, Artificial Sequence
  Description of Artificial Sequence Synthetic probe
cctccctcacgcgcctgcag

SEQ ID NO: 65 - DNA, 20 bp, Artificial Sequence
  Description of Artificial Sequence Synthetic probe
gtggactgagcgcggatggc

SEQ ID NO: 66 - DNA, 20 bp, Artificial Sequence
  Description of Artificial Sequence Synthetic probe
cgaggcagacgcgtcccacc

SEQ ID NO: 67 - DNA, 20 bp, Artificial Sequence
  Description of Artificial Sequence Synthetic probe
tggccgagactgcaggagcg

SEQ ID NO: 68 - DNA, 20 bp, Artificial Sequence
  Description of Artificial Sequence Synthetic probe
agcggacgactgcggacgag

SEQ ID NO: 69 - DNA, 20 bp, Artificial Sequence
  Description of Artificial Sequence Synthetic probe
gcctgcgaagacccaagcga

SEQ ID NO: 70 - DNA, 20 bp, Artificial Sequence
  Description of Artificial Sequence Synthetic probe
gagcagcgacgccgaggcag

SEQ ID NO: 71 - DNA, 20 bp, Artificial Sequence
  Description of Artificial Sequence Synthetic probe
gcgagtcccgagggtcccaa

SEQ ID NO: 72 - DNA, 20 bp, Artificial Sequence
  Description of Artificial Sequence Synthetic probe
ggaaagcgagcggcagccaa

SEQ ID NO: 73 - DNA, 19 bp, Artificial Sequence
  Description of Artificial Sequence Synthetic oligonucleotide
tggggcagagggactggta

SEQ ID NO: 74 - DNA, 57 bp, Artificial Sequence
  Description of Artificial Sequence Synthetic oligonucleotide
tggggcagagggactggtaacacgctggtaccattcgatatcgcctcctttagcggt

SEQ ID NO: 75 - DNA, 21 bp, Artificial Sequence
  Description of Artificial Sequence Synthetic oligonucleotide
accgctaaaggaggcgatatc

SEQ ID NO: 76 - DNA, 36 bp, Artificial Sequence
  Description of Artificial Sequence Synthetic oligonucleotide
ggaaagcgagcggcagccaaacacgctggtaccatt

(19) United States
(12) Patent Application Publication
(10) Pub. No.: US 2006/0024714 A1
(43) Pub. Date: Feb. 2, 2006

(54) Compositions and methods for detecting and quantitating polynucleotide sequences

(75) Inventor: Kai Qin Lao, Pleasanton, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 11/173,875

(22) Filed: Jun. 30, 2005

Related U.S. Application Data
(60) Provisional application No. 60/584,621, filed on Jun. 30, 2004.

(51) Int. Cl. C12Q 1/68; C12P 19/34

(52) U.S. Cl. 435/6; 435/91.2

(57) ABSTRACT

The present disclosure provides compositions and methods to detect and quantitate one or more target polynucleotide sequences.

This application claims under 35 U.S.C. § 119(e) the benefit of the filing date of U.S. Patent Application Ser. No. 60/584,621, filed Jun. 30, 2004, which is incorporated by reference in its entirety.

### 1. FIELD

This disclosure relates generally to compositions, methods and kits for analyzing nucleic acid sequences, and more specifically to compositions, methods and kits for detecting and/or quantitating polynucleotide sequences.

### 2. INTRODUCTION

Methods for detecting or quantitating target polynucleotide sequences, in most instances, amplify target sequences in the presence of reporter molecules that produce detectable signals proportional to the number of copies of amplified sequences, i.e. amplicons, that is produced. In these methods, the accumulation of amplicons is usually monitored in real-time by measuring the detectable signal produced at each successive amplification cycle. Although real-time monitoring of target sequence amplification is very sensitive and specific, the number of target sequences that can be simultaneously amplified and monitored in a multiplex format is limited. This is due in part to the limited number of reporter molecules currently available that produce distinguishable signals.

There is, accordingly, a need in the art for reporter molecules suitable for discriminating the amplicons produced from large numbers of target sequences amplified in a multiplex format.

### 3. SUMMARY

Disclosed herein are compositions and methods for analyzing one or more target sequences. In some embodiments, the methods comprise contacting a target sequence with a primer and a flap probe. The primer hybridizes to a target polynucleotide to form a substrate suitable for primer extension by a polymerase. The flap probe hybridizes to the target polynucleotide 3′ relative to the primer. A region of the flap probe does not hybridize to the target polynucleotide 3′ relative to the primer. A region of the flap probe does not hybridize to the target polynucleotide, thereby forming a single-stranded flap or cleavage sequence.

In some embodiments, hybridizing the primer and flap probe to a target polynucleotide forms a substrate for cleavage by the 5′-3′ nuclease activity of a polymerase, which releases the flap sequence. In some embodiments, the primer can be extended by the action of a polymerase. In some embodiments, cleavage of the flap probe can occur in a thermocycling reaction, which, in some embodiments, includes primer extension or template amplification.

The released flap sequence can be used to analyze, e.g., to detect and/or quantitate, a target polynucleotide. In some embodiments, the released flap sequence can be analyzed directly. In some embodiments, the released flap sequence can be further modified prior to analysis. Therefore, in various exemplary embodiments, a released flap sequence can be extended by a polymerase, ligated to a ligation partner, labeled with a moiety suitable for producing a detectable signal, or hybridized to one or more other polynucleotides, and the like.

In some embodiments, flap probes can be used to analyze a plurality of target polynucleotides in a multiplex format. Therefore, in some embodiments flap sequences of two or more probes can have varying degrees of similarity. In some embodiments, flap sequences of two or more probes can be identical. In some embodiments, the flap sequences of two or more probes can be substantially unique. Therefore, in some embodiments a flap sequence can be a code sequence.

In another aspect, the disclosure provides kits suitable for practicing the various embodiments of the disclosed methods. In some embodiments, a kit comprises one or more flap probes suitable for analyzing one or more target polynucleotides. In some embodiments, kits can include one or more other reagents, including but not limited to, one or more primers. The primers, flap probes and target polynucleotides are suitable to form a substrate for the 5′-3′ nuclease activity of a polymerase, which is capable of cleaving the flap sequence from the probe. In some embodiments, kits can comprise reagents for detecting or modifying a released flap sequence.

### 4. BRIEF DESCRIPTION OF THE DRAWINGS

The skilled artisan will understand that the drawings, described below, are for illustration purposes only. The drawings are not intended to limit the scope of the present disclosure in any way.

FIG. 1 provides a cartoon illustrating one embodiment of the disclosed methods wherein a cDNA target polynucleotide is exponentially amplified by forward and reverse amplification primers, and Taq DNA polymerase. The amplification is carried out in the presence of a flap probe hybridized to the target polynucleotide. The flap probe comprises a target specific sequence and a flap sequence. During each round of amplification Taq DNA polymerase cleaves the flap sequence from the probe. As amplification proceeds the number of released flap sequences increases proportionately. In this embodiment, the released flap sequences, which comprise a fluorescent moiety (F), are ligated to a ligation partner comprising a mobility modifier(s) (—O—O—) and detected.

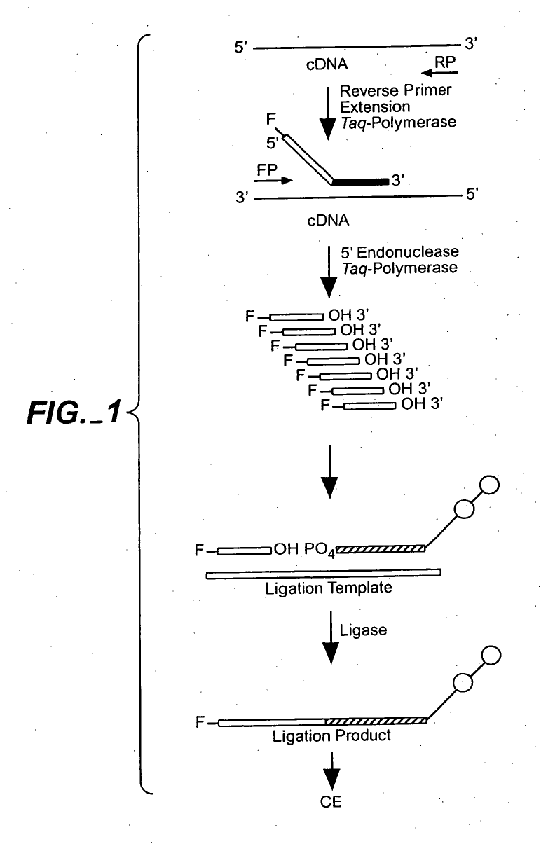

FIG. 2 provides a cartoon illustrating one embodiment of the disclosed methods, in which a flap probe comprises donor (Q)-acceptor (F) pair that provides a system suitable for monitoring the release of the flap sequence in real-time.

FIG. 3 provides a cartoon illustrating one embodiment of the disclosed methods wherein a 100 cDNA target polynucleotide and 100 control cDNA target polynucleotide are exponentially amplified by forward and reverse amplification primers, and Taq DNA polymerase. Each multiplex amplification reaction is carried out in the presence of the same probes hybridized to the target polynucleotides. The released flap sequences (RFS) in each reaction are ligated to ligation partners (LP) each having different mobility modifiers to form up to 200 distinguishable ligation products. Therefore, the ligation products formed in each reaction may be co-electrophoresed for detection and quantitation.

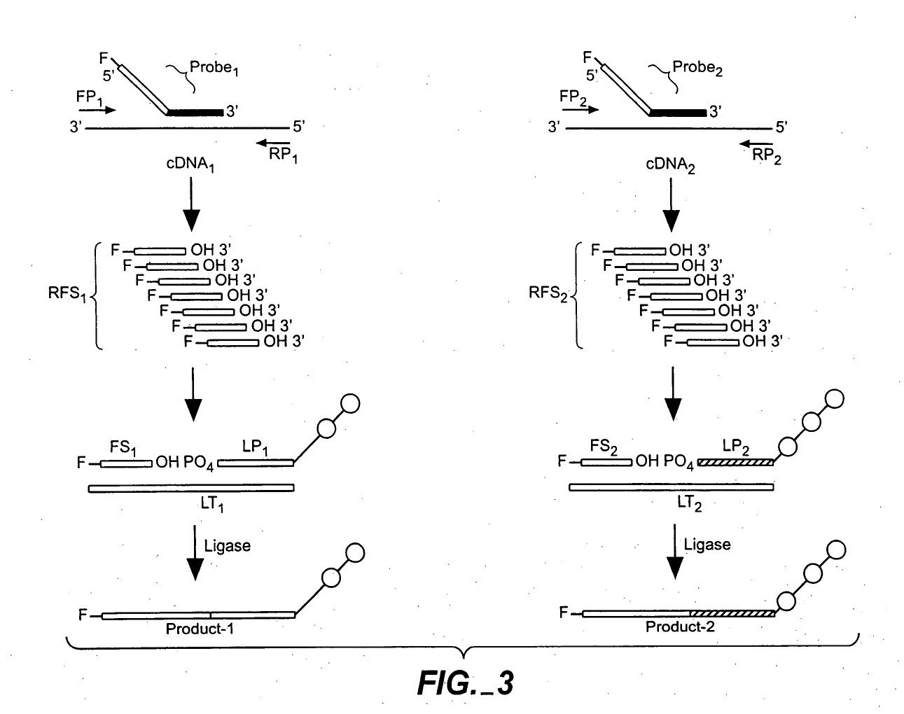

FIG. 4 illustrates the results of one embodiment of the disclosed methods wherein 12 synthetic polynucleotide sequences were ligated to 12 different ligation partners in a multiplex format and analyzed by capillary electrophoresis. The concentration of the synthetic polynucleotide utilized is shown in Panels A-D.

FIG. 5 illustrates the results of one embodiment of the disclosed methods wherein a COX6b synthetic target sequence is amplified by forward and reverse amplification primers and Taq DNA polymerase. The concentration of synthetic target sequence utilized is indicated in Panels A-D. Panel E the results of the reaction described in FIG. 4 utilized as a control.

### 5. DETAILED DESCRIPTION

It is to be understood that both the foregoing general description, including the drawings, and the following detailed description are exemplary and explanatory only and are not restrictive of this disclosure. In this disclosure, the use of the singular includes the plural unless specifically stated otherwise. Also, the use of “or” means “and/or” unless stated otherwise. Similarly, “comprise,” “comprises,” “comprising” “include,” “includes,” and “including” are not intended to be limiting.

This disclosure provides methods, compositions and kits for detecting or quantitating polynucleotide sequences.

As discussed in the Summary section, in some embodiments the disclosed methods comprise contacting a polynucleotide comprising a target sequence (“target polynucleotide”) with a primer, a probe and a polymerase having 5′-3′ nuclease activity. In some embodiments, the primer comprises a nucleobase sequence suitable for amplifying the target sequence. In some embodiments, the probe can be a “flap” probe comprising at least two domains or regions. One flap probe domain comprises a nucleobase sequence suitable for hybridizing to the target polynucleotide at a position 3′ relative to the primer. Another flap probe domain comprises a nucleobase sequence that is not suitable for hybridizing to the target polynucleotide. Therefore, when a flap probe hybridizes to its target polynucleotide, one domain of the probe forms one strand of a double-stranded nucleic acid and another domain forms a single-stranded region or “flap” sequence.

In some embodiments, the flap probe, primer and target polynucleotide hybridize under conditions suitable to form a substrate for the nuclease activity of the polymerase, which cleaves the unhybridized flap sequence from the nuclease probe. In some embodiments, thermocycling is employed to form additional substrates for the nuclease activity of the polymerase.

In some embodiments, a substrate for the 5′-3′ nuclease activity may be formed by the hybridization of the flap probe and primer to the target polynucleotide under conditions suitable for extension of the primer by the polymerase. Therefore, in some embodiments, the release of the flap sequence may occur during an amplification reaction, which, in some embodiments, comprises multiple rounds of thermocycling.

Regardless of the method employed, the released flap sequence may be utilized as an indicator of the presence, composition and/or quantity of one or more target polynucleotides. Therefore, in various embodiments, the released flap sequence may be directly detected or quantitated or may be modified prior to detection or quantitation.

Furthermore, in some embodiments, multiples sets of primers and flap probes may be designed to simultaneously detect or quantitate a plurality of target polynucleotide sequences in multiplex formats.

As used herein, “probe” refers to a nucleobase polymer suitable for hybridizing to a target polynucleotide (“target specific sequence”). As used herein, “flap probe,” and “cleavage probe” refer to a nucleobase polymer suitable for hybridizing to a target polynucleotide and for providing a “flap” or “cleavage” sequence suitable for release by the 5′-3′ nuclease activity of a polymerase. The released flap sequence is suitable for analysis by the disclosed methods, as described below. The skilled artisan will appreciate that the definition of flap probe provided herein, differs from a “linear probe” or “conventional probe” which when hybridized to its complementary sequence does not provide a “flap” or “cleavage” sequence suitable for release by the 5′-3′ activity of a polymerase and modification or detection as described below. Flap probes may be designed to have target specific and flap sequences in any orientation; therefore, in some embodiments, the flap sequence can be 5′ relative to the target specific sequence, and, in some embodiments, the flap can be 3′ relative to the target specific sequence.

The target specific sequences of the flap probes may be designed to be substantially complementary to the target sequence or to a region of the target polynucleotide that flanks the target sequence. Thus, the actual nucleobases that comprise each target specific sequence may depend upon the number and type of target sequences and target polynucleotides, which will be apparent to those of skill in the art.

Generally, each target specific sequence should be sufficiently long to hybridize to the target polynucleotide under the conditions of the disclosed methods. By “hybridizing” or “annealing” is meant base-pairing interactions of one nucleobase polymer with another that results in the formation of a double-stranded structure. In some embodiments, annealing occurs via Watson-Crick base-pairing interactions, but may be mediated by other hydrogen-bonding interactions, such as Hoogsteen base pairing.

The exact length of the target specific sequence may depend on many factors, including but not limited to, the desired hybridization temperature between the target specific sequences and target polynucleotides, the complexity of the different target polynucleotides to be analyzed, the salt concentration, ionic strength, pH and other buffer conditions. The ability to select lengths and sequences suitable for hybridization is within the abilities of ordinarily skilled artisans (see, e.g., Sambrook et al. Molecular Cloning: A Laboratory Manual 9.50-9.51, 11.46, 11.50 (2d. ed., Cold Spring Harbor Laboratory Press); Sambrook et al., Molecular Cloning: A Laboratory Manual 10. 1-10.10 (3d. ed. Cold Spring Harbor Laboratory Press)). In some embodiments, the target specific sequence contains from about 15 to about 35 nucleotides, although in some embodiments the target specific sequences may contain more or fewer nucleotides. Shorter target specific sequences generally require lower temperatures to form sufficiently stable hybrid complexes with target polynucleotides. The capability of target specific sequences to anneal can be determined by the melting temperature (“Tₘ”) of the hybrid complex. Tₘ is the temperature at which 50% of a polynucleotide strand and its perfect complement form a double-stranded polynucleotide. In some embodiments, in which a target sequence is amplified by successive rounds of thermocycling, the target specific sequences should be designed to have a melting temperature (“Tₘ”) in the range of about 60-75° C. Melting temperatures in this range tend to insure that the probes remain annealed or hybridized to the target polynucleotide at the initiation of amplification, e.g., during primer extension. In addition, melting temperatures in this range also tend to insure that the probes remain annealed to the target polynucleotide at a temperature that it suitable for the 5′-3′ nuclease activity of a thermostable polymerase.

In some embodiments, wherein a plurality of flap probes are simultaneously utilized in a multiplex format, the melting temperatures of the various target specific sequences can be different; however, in an alternative embodiment they can be approximately the same, e.g., the Tₘ of each target specific sequence can be within a range of about 5° C. or less. The Tₘs of various target specific sequences can be determined empirically utilizing melting techniques that are well-known in the art (see, e.g., Sambrook et al. Molecular Cloning: A Laboratory Manual 11.55-11.57 (2d. ed., Cold Spring Harbor Laboratory Press)). Alternatively, the Tₘ of a target specific sequence can be calculated. Numerous references and aids for calculating Tₘs are available in the art and include, by way of example and not limitation, Baldino et al. Methods Enzymology. 168:761-777; Bolton et al., 1962, Proc. Natl. Acad. Sci. USA 48:1390; Bresslauer et al., 1986, Proc. Natl. Acad. Sci. USA 83:8893-8897; Freier et al., 1986, Proc. Natl. Acad. Sci. USA 83:9373-9377; Kierzek et al., Biochemistry 25:7840-7846; Rychlik et al., 1990, Nucleic Acids Res. 18:6409-6412 (erratum, 1991, Nucleic Acids Res. 19:698); Rychlik. J. NIH Res. 6:78; Sambrook et al. Molecular Cloning: A Laboratory Manual 9.50-9.51, 11.46-11.49 (2d. ed., Cold Spring Harbor Laboratory Press); Sambrook et al., Molecular Cloning: A Laboratory Manual 10. 1-10.10 (3d. ed. Cold Spring Harbor Laboratory Press)); Suggs et al., 1981, In Developmental Biology Using Purified Genes (Brown et al., eds.), pp. 683-693, Academic Press; Wetmur, 1991, Crit. Rev. Biochem. Mol. Biol. 26:227-259, which disclosures are incorporated by reference. Any of these methods can be used to determine a Tₘ of a target specific sequence.

Thus, in some embodiments, a target specific sequence is substantially complementary or suitable for hybridizing to a target polynucleotide. “Substantially complementary” and “suitable for hybridizing” refers to a sequence that includes enough complementary to hybridize to another sequence at the concentration and under the temperature and conditions employed. Therefore, in some embodiments, a target specific sequence may be completely complementary to a target polynucleotide and, in some embodiments it may be desirable to include one or more nucleotides of mismatch or non-complementarity, as is well known in the art. For example, in some embodiments, wherein the target specific sequence comprises the 3′ region of a flap probe, the 3′ terminal nucleotide may be a region of mismatch relative to the target polynucleotide to inhibit extension of the flap probe by the action of a polymerase. Therefore, “regions of mismatch,” “non-complementarity, ” and “region of sequence diversity” refer to at least one nucleotide of a polynucleotide sequence that is not suitable for base-pairing with another polynucleotide sequence. Therefore, the term “region of mismatch” is used when comparing sequences, such as, primer sequences; a primer sequence and a target sequence; a probe sequence and a target sequence; and the like.

In contrast to the target specific sequences, the flap or cleavage sequences are designed to be substantially non-complementary to the target polynucleotides. Therefore, the flap sequences are regions of mismatch relative to the target polynucleotides. The actual nucleobases that comprise each flap sequence may depend upon the number and type of target sequences and target polynucleotides to be analyzed, the assay conditions (e.g., temperature, pH, ionic strength, etc.), and the extent to which each flap sequence may be discriminated. Therefore, in some embodiments, each flap sequence may be substantially unique. By “substantially unique” is meant the sequence is suitable to identify or distinguish the sequence from at least one other polynucleotide sequence employed in a disclosed method. As described above, a flap sequence is not suitable for hybridizing to a target polynucleotide, and, therefore, relative to a target polynucleotide a flap sequence can be substantially unique. However, in some embodiments, a flap sequence may be substantially unique in comparison to the flap sequence of at least one other probe. Therefore, in some embodiments, a flap sequence comprises a code sequence. By “code sequence” is meant a flap sequence that is substantially unique in comparison to other flap sequences employed in a disclosed method. The use of code sequences in various types of nucleic acid assays and reactions are known in the art (see, e.g., U.S. Pat. Nos. 5,314,809, 5,853,989, 6,090,552, 6,355,431, the disclosures of which are incorporated by reference). In some embodiments a code sequence may have no statistically significant sequence homology or identity with other flap sequences. However, a skilled artisan will appreciate that in some embodiments a single nucleotide in a flap sequence may be sufficient for a flap sequence to be a code sequence. In one non-limiting example, a flap sequence may be a code sequence by being one nucleotide longer or shorter than another flap sequence. In some embodiments, such flap sequences may be differentiated by electrophoretic techniques, including but not limited to, capillary electrophoresis. Thus, the suitability of a particular sequence to be a code sequence may depend, at least in part, upon the technique employed to analyze or detect the released flap sequence.

In some embodiments, a flap sequence may not be substantially unique and, therefore, may have statistically significant sequence homology to the flap sequence of another probe. Therefore, in some embodiments, two or more flap sequences may be identical in length and/or composition. Embodiments in which such flap sequences find use include, but are not limited to, assays in which a sample is screened for the presence or absence of one or more target polynucleotides. In such embodiments, discrimination of the released flap sequences and, therefore, the various target polynucleotides is generally not desired.

Once released, the flap sequences may be detected by various techniques as known in the art. In some embodiments, a released flap sequence may be detected directly without modification. Therefore, in some embodiments the released flap sequence is detected in the form in which is it released from the probe. In various non-limiting examples, a released flap sequence may be directly detected by capillary electrophoresis (see, e.g., U.S. Pat. Nos. RE37,941, 6,372,106, 6,372,484, 6,387,234, 6,387,236, 6,402,918, 6,402,919, 6,432,651, 6,462,816, 6,475,361, 6,476,118, 6,485,626, 6,531,041, 6,544,396, 6,576,105, 6,592,733, 6,596,140, 6,613,212, 6,635,164, 6,706,162) or by array-based assays (see, e.g., U.S. Pat. Nos. 5,405,783, 5,445,934, 5,510,270, 5,547,839, 6,232,062, 6,221,583, 6,309,822, 6,344,316, 6,355,431, 6,355,432, 6,368,799, 6,396,995, 6,410,229, 6,440,667, 6,576,425, 6,576,424 6,600,031, 6,632,605, 6,646,243, 6,495,323, 6,667,394, 6,670,122, 6,686,150). However, the skilled artisan will appreciate that, in some embodiments, a modified flap sequence, as described below, may be detected by methods suitable for detecting an unmodified flap sequence.

In some embodiments, a released flap sequence may be modified prior to detection. For example, in some embodiments, the released flap sequence may comprise the ligand of a binding partner or an anti-ligand. Therefore, in some embodiments, a flap sequence is modified by the binding of the binding partner to the ligand. Thus, “ligand,” “binding partner” and “anti-ligand” as used herein refer to molecules that specifically interact with each other. “Specifically interact” refers to binding that is substantially distinctive and restricted, and sufficient to be sustained under conditions that inhibit non-specific binding. Non-limiting examples of ligand binding include but are not limited to antigen-antibody binding (including single-chain antibodies and antibody fragments (e.g. Fab, Fab′, F(ab′)₂, Fv)), hormone-receptor binding, neurotransmitter-receptor binding, polymerase-promoter binding, substrate-enzyme binding, allosteric effector-enzyme binding, biotin-streptavidin binding, digoxin-anti-digoxin binding, carbohydrate-lectin binding, or a molecule that donates or accepts a pair of electrons to form a coordinate covalent bond with the central metal atom of a coordination complex. In various exemplary embodiments, the dissociation constant of the ligand/anti-ligand complex is less than about 10⁻⁴-10⁻⁹ M⁻¹, less than about 10⁻⁵-10⁻⁹ M⁻¹ or less than about 10⁻⁷-10⁻⁹ M⁻¹. In some embodiments, a ligand and/or binding partner comprise one or more detectable moieties, described below.

In some embodiments, a released flap sequence is modified by the action of one or more enzymes. Non-limiting examples of enzymes suitable for modifying a released flap sequence include polymerases (e.g., DNA-directed DNA polymerases, RNA-directed DNA polymerases, terminal transferases, thermostable polymerases (e.g., Taq, Pfu, Vent), reverse transcriptases, Klenow fragment, T4 DNA polymerase, T7 DNA polymerase), ligases (e.g., thermostable ligases, T4 DNA ligase), polynucleotide kinases, phosphatases (e.g., bacterial alkaline phosphatase, calf intestinal alkaline phosphatase, shrimp alkaline phosphatase), endonucleases (e.g., restriction endonucleases I-III), and exonucleases (e.g., exonucleases I-III, mung bean nuclease, BAL31 nuclease, S1 nuclease). Therefore, in various exemplary embodiments, a released flap sequence may be modified by the addition or removal of nucleotides or phosphate groups, by ligation to another polynucleotide, by cleavage of the flap sequence, by the addition or removal of a moiety (e.g., a ligand or a moiety suitable for producing a detectable signal, as described below), or by amplification of the released flap sequence (e.g., by PCR, LCR, LDR, OLA). In some embodiments, a released flap sequence may be suitable to initiate a coupled amplification reaction (see, e.g., U.S. Patent Application Ser. No. 60/584,665, filed Jun. 30, 2004).

The skilled artisan will appreciate that in some embodiments modification or detection of a released flap sequence may include hybridizing the released flap sequence to another polynucleotide, such as, a primer, a linear probe, or template (e.g., a polymerization template or ligation template). When hybridized to another polynucleotide, the released flap sequence, in various embodiments, may itself function as a linear probe, template, primer and/or a substrate (e.g., a ligation partner, as describe below). Therefore, in some embodiments, a flap sequence may be designed to be substantially complementary to a polynucleotide that is used in methods of detecting the released flap sequence. In some embodiments, wherein a released flap sequence is ligated to a ligation partner, the flap sequence and ligation partner are hybridized to a ligation template under conditions suitable for a ligase to form a covalent bond between the 3′-hydroxyl of one sequence and the 5′-phosphate of the other polynucleotide. Thus, in some embodiments, the resulting “ligation product” may be formed by ligating the flap sequence to the 3′ or 5′ terminus of the ligation partner. In some embodiments, the conditions suitable for ligation may include thermocycling in the presence of a thermostable ligase to form a “ligation amplicon”. In some embodiments, the flap and ligation partners, when hybridized to the ligation template, may be separated by a gap of at least one nucleotide and, therefore, are not suitable for ligation. Therefore, in some embodiments, the sequence hybridized to the ligation template 5′ relative to the other sequence may be extended by the action of a polymerase. In some embodiments, a gap between the hybridized flap and ligation partner may be filled-in by hybridizing a second ligation partner to the ligation template.

Generally, each released flap sequence should be sufficiently long and comprise sequence sufficient for its detection or quantitation by the method selected by the practitioner. Similarly, the polynucleotides employed to detect or quantitate a released flap sequence also should be sufficiently long and comprise a sequence suitable for detecting or modifying the released flap sequence. Factors to be considered in selecting the length and composition of a flap sequence and the polynucleotides employed in its detection or modification include but are not limited to, the method of detection, the efficiency of a reaction selected to modify the released flap sequence, the number of types of polynucleotides employed to detect the released flap sequences, the conditions under which the flap sequence is released, the presence or absence of moieties on the released flap sequence (e.g., ligands or detectable moieties), the complexity of the different target polynucleotides to be analyzed, the complexity of the different flap sequences, and the reaction conditions (e.g., temperature, salt concentration, ionic strength, pH, and the like). The ability to design flap sequences and polynucleotides of suitable length and composition is within the capabilities of ordinarily skilled artisans (see, e.g., Sambrook et al. Molecular Cloning: A Laboratory Manual 9.50-9.51, 11.46, 11.50 (2d. ed., Cold Spring Harbor Laboratory Press); Sambrook et al., Molecular Cloning: A Laboratory Manual 10.1-10.10 (3d. ed. Cold Spring Harbor Laboratory Press)). However, generally, flap sequences comprise from about 15 to about 35 nucleotides, although in some embodiments the flap sequences may contain more or fewer nucleotides/nucleobases. Furthermore, polynucleotides employed to detect or modify a released flap sequence may be shorter or longer than the flap sequence. As described above, the capability of sequences to anneal can be determined by the melting temperature (“Tₘ”) of the hybrid complex. Thus, the factors described herein in the design of target specific sequences suitable for hybridizing to a target polynucleotide are, in some embodiments, applicable to the design of flap sequences and polynucleotides used for their detection or modification.

In some embodiments, wherein the flap sequence is released from a probe by the 5′-3′ nuclease activity of a polymerase, the probe is hybridized to a target sequence 3′ relative to a primer. By “primer” herein is meant a polynucleotide capable of hybridizing or annealing to a template polynucleotide to form a substrate for a polymerase that synthesizes polynucleotides in a template directed manner (e.g., DNA-dependent DNA polymerases, RNA-dependent DNA polymerase (reverse transcriptase)). Therefore, in various exemplary embodiments, a primer can be an exponential amplification primer, a reverse transcription primer, and/or a linear primer. By “exponential amplification primer” as used herein is meant a primer suitable for exponentially amplifying a target sequence. In exponential target sequence amplification, the product of each amplification cycle is an amplicon that is a suitable template for subsequent amplification cycles. Therefore, as known in the art, exponential amplification generally utilizes at least two exponential primers. For example, PCR-based amplification generally utilizes a pair of “forward” and “reverse” primers. Therefore, the skilled artisan is aware that the suitability of a primer for exponential amplification depends, in part, on the presence of a second suitable primer. The forward and reverse primers hybridize to a target sequence in opposite orientations to produce complementary DNA strands to form double-stranded amplicons that serve as templates for further rounds of amplification.

In some embodiments, a primer is a linear primer. By “linear amplification primer” herein is meant a primer suitable to linearly amplify a polynucleotide sequence. In linear target sequence amplification, the product of each amplification cycle is not suitable for subsequent amplification cycles. For example, the linear amplification of a target sequence generally produces a single-stranded amplicon that does not hybridize to the linear primer and, therefore, is not a suitable template for subsequent amplification cycles.

The primers may be target sequence-specific or may be designed to hybridize to sequences that flank a target sequence to be amplified. In some embodiments, a primer may hybridize to a sequence introduced into the amplicon during the amplification reaction. Thus, the actual nucleotide sequences of each primer may depend upon the target sequence, target polynucleotide, and conditions under which the target sequence may be amplified, which will be apparent to those of skill in the art. Methods for designing primers suitable for amplifying target sequences of interest are well-known (see, e.g., Dieffenbach et al., General Concepts for PCR Primer Design, in PCR Primer, A Laboratory Manual, Dieffenbach, C. W, and Dveksler, G. S., Ed., Cold Spring Harbor Laboratory Press, New York, 1995, 133-155; Innis, M. A. et al. Optimization of PCRs, in PCR protocols, A Guide to Methods and Applications, Innis, M. A., Gelfand, D. H., Sninsky, J. J., and White, T. J., Ed., CRC Press, London, 1994, 5-11; Sharrocks, et al. The design of primers for PCR, in PCR Technology, Current Innovations, Griffin, H. G., and Griffin, A. M, Ed., CRC Press, London, 1994, 5-11; Suggs et al., Using Purified Genes, in ICN-UCLA Symp. Developmental Biology, Vol. 23, Brown, D. D. Ed., Academic Press, New York, 1981, 683; Kwok et al. Effects of primer-template mismatches on the polymerase chain reaction: Human Immunodeficiency Virus 1 model studies. Nucleic Acids Res. 18:999-1005, 1990; Compton T (1990). Degenerate primers for DNA amplification. pp. 39-45 in: PCR Protocols (Innis, Gelfand, Sninsky and White, eds.); Academic Press, NY; Fuqua et al. (1990). BioTechniques 9(2):206-211; Gelfand et al., 1990, Thermostable DNA polymerases. pp. 129-141 in: PCR Protocols (Innis, Gelfand, Sninsky and White, eds.); Academic Press, NY; Innis et al., 1990, Optimization of PCRs. pp. 3-12 in: PCR Protocols (Innis, Gelfand, Sninsky and White, eds.); Academic Press, NY; Krawetz et al., 1989, Nucleic Acids Research 17(2):819; Rybicki et al., 1990, Journal of General Virology 71:2519-2526; Rychlik et al., 1990, Nucleic Acids Research 18(21):6409-6412; Sarkar et al., 1990, Nucleic Acids Research 18(24):7465; Smith et al., 1990, 9/90(5):16-17; Thweatt et al. 1990, Analytical Biochemistry 190:314-316; Wu et al., 1991, DNA and Cell Biology 10(3):233-238; Yap et al., 1991, Nucleic Acids Research 19(7):1713, which provide examples demonstrating how particular primer pairs may be designed).

The factors considered in the design of the target specific sequences and flap sequences of the probes, described above, also are generally applicable to the design of primer sequences. However, an additional factor to be considered is that the 3′ terminus of a primer when hybridized to a target polynucleotide is suitable for template directed extension by a polymerase. Therefore, the ₃′ terminus of a primer is generally complementary to the target polynucleotide.

In some embodiments, another factor to consider in designing a primer is the structure of the primer and target polynucleotide. As the skilled artisan will appreciate, in general, the relative stability and therefore, the Tₘs, of RNA:RNA, RNA:DNA, and DNA:DNA hybrids having identical sequences for each strand may differ. In general, RNA:RNA hybrids are the most stable (highest relative Tₘ) and DNA:DNA hybrids are the least stable (lowest relative Tₘ). For example, in the embodiment in which an RNA target polynucleotide is reverse transcribed to produce a cDNA, the determination of the suitability of a DNA primer for the reverse transcription reaction can include the effect of the RNA polynucleotide on the Tₘ of the primer. Although the Tₘs of various hybrids may be determined empirically, as described above, examples of methods of calculating the Tₘ of various hybrids are found at Sambrook et al. Molecular Cloning: A Laboratory Manual 9.51 (2d. ed., Cold Spring Harbor Laboratory Press).

Although in some embodiments the sequences of the primers may be substantially or completely complementary to a target polynucleotide, in some embodiments a primer may include one or more regions of mismatch or non-complementarity. In some embodiments, the regions of mismatch and/or their complementary sequences are incorporated into the amplicons by target sequence amplification to provide useful cites for downstream hybridization or amplification reactions, as described below. Determining the number, type, length, and composition of regions of mismatch, their position within a primer and their distribution or commonality among the various primers are within the capabilities of the ordinary skilled artisan. In some embodiments, a primer sequence that is a region of mismatch in comparison to a target sequence is substantially unique to that primer. Therefore, in some embodiments, a primer comprises a code sequence, as described above. In some embodiments, a region of mismatch between a primer and a target sequence is a sequence that is shared by more than one primer. Thus, in various exemplary embodiments, a “shared sequence” may be common to each forward primer, each reverse primer, or each linear primer. Thus, by “forward universal sequence” and “reverse universal sequence” are meant a primer sequence of continuous nucleotides that is a region of diversity in comparison to a target sequence that is shared by each forward or reverse primer. Primers and methods for amplifying sequences to introduce regions of mismatch into the amplified sequences are known in the art (see, e.g., U.S. Pat. Nos. 5,314,809, 5,853,989, 5,882,856, 6,090,552, 6,355,431, 6,617,138, 6,630,329, 6,635,419, 6,670,130, 6,670,161 and Weighardt et al., 1993, PCR Methods and App. 3:77, the disclosures of which are incorporated by reference). Once incorporated into an amplicon, the regions of mismatch or their complementary sequence may be suitable for hybridizing to a primer or probe. Thus, in some embodiments, the incorporated sequences or their complement may be suitable to hybridize to primers for subsequent rounds of amplification. In some embodiments, an incorporated sequence or its complement may be suitable for hybridizing to a target specific sequence of a flap probe. Therefore, in some embodiments, a flap probe may hybridize to a sequence or its complement that is incorporated into an amplicon by the amplification of a target sequence.

To provide a substrate for the 5′-3′ nuclease activity of a polymerase a flap probe hybridizes to a target polynucleotide 3′ relative to a primer. Non-limiting examples of polymerases with 5′-3′ nuclease activity include, but are not limited to, AmpliTaq® DNA polymerase, AmpliTaq-GOLD®, AmpliTaq® FS (Applied Biosystems, Foster City, Calif.), E. coli DNA polymerase I (New England Biolabs, Beverly, Mass.), rBst DNA Polymerase (Epicenter®, Madison, Wis.), and Tf1 DNA polymerase (Promega Corp., Madison, Wis.). The nuclease activity of the polymerase and its capability to release the flap sequence is, at least in part, influenced by the distance between the 3′ terminus of the primer and the most 5′ nucleobase of the flap probe that is hybridized to the target sequence. Therefore, in some embodiments, extension of the primer by the polymerase may not be required for the flap probe to be released. For example, if the 3′ terminus of the primer is at least within about 20 nucleobases of the 5′ hybridized nucleobase of the flap probe, primer extension and, therefore, target sequence amplification may not be required for release of the flap sequence. In embodiments wherein the distance between the primer and flap probe is greater than about 20 nucleobases, extension of the primer may be required for release of the flap sequence from a probe. Therefore, in some embodiments, the primer may be extended such that its 3′ terminus is within at least about 20 nucleobases of the hybridized flap probe. In some embodiments, the primer may be extended to amplify the target sequence. Therefore, in some embodiments, release of the flap sequence may occur during amplification of the target sequence.

The concentrations of the various polynucleotides disclosed herein (e.g., probes, primers, templates, ligation partners, and the like) may vary widely. However, in some embodiments, the concentrations of the various polynucleotides are generally non-limiting. By “non-limiting” herein is meant a concentration that does not limit the rate of the detection or quantitation of a target polynucleotide. Therefore, in various embodiments, the concentration of any given polynucleotide is in excess of the target sequence, is in excess of the number of amplicons produced from the target sequence, and/or is in excess of the number of released flap sequences. The skilled artisan is aware that as the detection or quantitation of a target nucleic acid proceeds by the disclosed methods one or more polynucleotides may eventually be consumed. Therefore, the concentrations of the polynucleotides are at least non-limiting at the commencement of the reaction selected to detect or quantitate a target polynucleotide. Determining an appropriate concentration for each polynucleotide employed is within the abilities of the skilled artisan. For example, the skilled artisan will appreciate that in some embodiments the concentration of two or more polynucleotides can be substantially equivalent (e.g., forward and reverse amplification primers), whereas in other embodiments, substantially equivalent concentrations are not required and may be advantageously avoided. Therefore, in various exemplary embodiments, the concentration of any given polynucleotide may be at least about 100 nM, at least about 500 nM, to at least about 1 μM, or even greater.

The various polynucleotides described herein may be of any chemical composition that is suitable for the polynucleotide to carry out its intended function. Thus, in one non-limiting example, a flap probe may be of any chemical composition suitable for hybridizing to a target polynucleotide and for providing a “flap” suitable for release by the 5′-3′ nuclease activity of a polymerase under the conditions of the disclosed methods. Therefore, in some embodiments, a flap probe may comprise nucleobases that are substantially resistant to the 5′-3′ nuclease activity of a polymerase with the exception of a sequence within the flap probe that is to be cleaved by the nuclease activity. In another non-limiting example, a primer may be of any chemical composition suitable for hybridizing to a template and for providing a substrate for template directed primer extension by the action of a polymerase. In another non-limiting example, a template may be of any chemical composition suitable for hybridizing to one or more primers or probes and to form a substrate for a polymerase or ligase. Determining the types of nucleobase polymers suitable for the function of each polynucleotide is within the abilities of the skilled artisan.

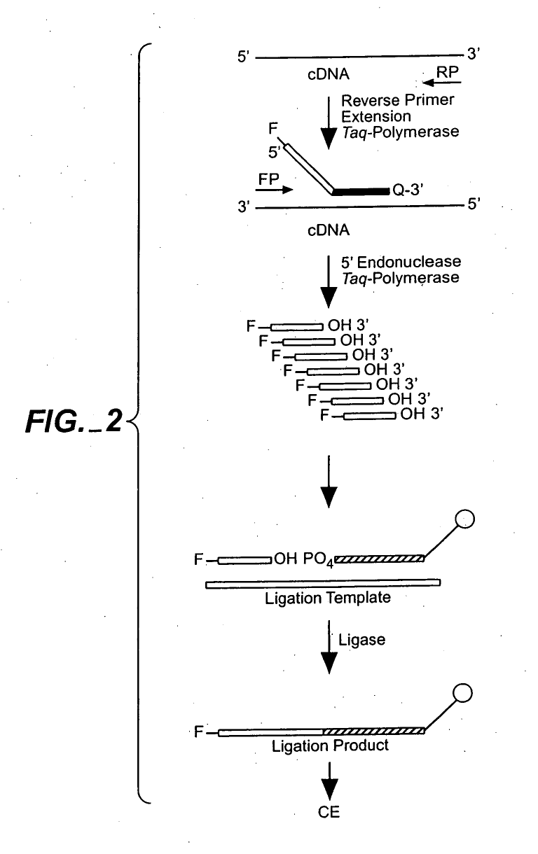

Therefore, by “nucleobase” is meant naturally occurring and synthetic heterocyclic moieties commonly known to those who utilize nucleic acid or polynucleotide technology or utilize polyamide or peptide nucleic acid technology to generate polymers that can hybridize to polynucleotides in a sequence-specific manner. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobases include those nucleobases disclosed in FIGS. 2(A) and 2(B) of Buchardt et al. (WO 92/20702, WO 92/20703, and U.S. Pat. No. 6,357,163).

Nucleobases can be linked to other moieties to form nucleosides, nucleotides, and nucleoside/tide analogs. As used herein, “nucleoside” refers to a compound consisting of a purine, deazapurine, or pyrimidine nucleoside base, e.g., adenine, guanine, cytosine, uracil, thymine, 7-deazaadenine, 7-deazaguanosine, that is linked to the anomeric carbon of a pentose sugar at the 1′ position, such as a ribose, 2′-deoxyribose, or a 2′,3′-di-deoxyribose. When the nucleoside base is purine or 7-deazapurine, the pentose is attached at the 9-position of the purine or deazapurine, and when the nucleoside base is pyrimidine, the pentose is attached at the 1-position of the pyrimidine. (see, e.g., Komberg and Baker, DNA Replication, 2nd Ed. (Freeman, San Francisco, 1992)) The term “nucleotide” as used herein refers to a phosphate ester of a nucleoside, e.g., a mono-, a di-, or a triphosphate ester, wherein the most common site of esterification is the hydroxyl group attached to the C-5 position of the pentose. “Nucleotide 5′-triphosphate” refers to a nucleotide with a triphosphate ester group at the 5′ position. The term “nucleoside/tide” as used herein refers to a set of compounds including both nucleosides and nucleotides.

“Nucleobase polymer or oligomer” refers to two or more nucleobases connected by linkages that permit the resultant nucleobase polymer or oligomer to hybridize to a polynucleotide having a complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyamide or peptide nucleic acids. Nucleobase polymers or oligomers can vary in size from a few nucleobases, from 2 to 40 nucleobases, to several hundred nucleobases, to several thousand nucleobases, or more.

“Polynucleotide or oligonucleotide” refers to nucleobase polymers or oligomers in which the nucleobases are connected by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof.

In some embodiments, a nucleobase polymer is a polynucleotide analog or an oligonucleotide analog. By “polynucleotide analog or oligonucleotide analog” is meant nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. No. 6,013,785 and U.S. Pat. No. 5,696,253 (see also, Dagani, 1995, Chem. & Eng. News 4-5:1153; Dempey et al., 1995, J. Am. Chem. Soc. 117:6140-6141). Such positively charged analogues in which the sugar is a 2′-deoxyribose are referred to as “DNGs,” whereas those in which the sugar is ribose are referred to as “RNGs.” Specifically included within the definition of poly- and oligonucleotide analogs are locked nucleic acids (LNAs; see, e.g., Elayadi et al., 2002, Biochemistry 41:9973-9981; Koshkin et al., 1998, J. Am. Chem. Soc. 120:13252-3; Koshkin et al., 1998, Tetrahedron Letters, 39:4381-4384; Jumar et al., 1998, Bioorganic & Medicinal Chemistry Letters 8:2219-2222; Singh and Wengel, 1998, Chem. Commun., 12:1247-1248; WO 00/56746; WO 02/28875; and WO 01/48190.

In some embodiments, a nucleobase polymer is a polynucleotide mimic or oligonucleotide mimic. By “polynucleotide mimic or oligonucleotide mimic” is meant a nucleobase polymer or oligomer in which one or more of the backbone sugar-phosphate linkages is replaced with a sugar-phosphate analog. Such mimics are capable of hybridizing to complementary polynucleotides or oligonucleotides, or polynucleotide or oligonucleotide analogs or to other polynucleotide or oligonucleotide mimics, and may include backbones comprising one or more of the following linkages: positively charged polyamide backbone with alkylamine side chains as described in U.S. Pat. Nos. 5,786,461, 5,766,855, 5,719,262, 5,539,082 and WO 98/03542 (see also, Haaima et al., 1996, Angewandte Chemie Int′l Ed. in English 35:1939-1942; Lesnick et al., 1997, Nucleosid. Nucleotid. 16:1775-1779; D'Costa et al., 1999, Org. Lett. 1:1513-1516; Nielsen, 1999, Curr. Opin. Biotechnol. 10:71-75); uncharged polyamide backbones as described in WO 92/20702 and U.S. Pat. No. 5,539,082; uncharged morpholino-phosphoramidate backbones as described in U.S. Pat. Nos. 5,698,685, 5,470,974, 5,378,841 and 5,185,144 (see also, Wages et al., 1997, BioTechniques 23:1116-1121); peptide-based nucleic acid mimic backbones (see, e.g., U.S. Pat. No. 5,698,685); carbamate backbones (see, e.g., Stirchak and Summerton, 1987, J. Org. Chem. 52:4202); amide backbones (see, e.g., Lebreton, 1994, Synlett. February, 1994:137); methylhydroxyl amine backbones (see, e.g., Vasseur et al., 1992, J. Am. Chem. Soc. 114:4006); 3′-thioformacetal backbones (see, e.g., Jones et al., 1993, J. Org. Chem. 58:2983) and sulfamate backbones (see, e.g., U.S. Pat. No. 5,470,967). All of the preceding references are herein incorporated by reference.

“Peptide nucleic acid” or “PNA” refers to poly- or oligonucleotide mimics in which the nucleobases are connected by amino linkages (uncharged polyamide backbone) such as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112 and 6,403,763; all of which are incorporated herein by reference. The term “peptide nucleic acid” or “PNA” shall also apply to any oligomer or polymer comprising two or more subunits of those polynucleotide mimics described in the following publications: Lagriffoul et al., 1994, Bioorganic & Medicinal Chemistry Letters, 4:1081-1082; Petersen et al., 1996, Bioorganic & Medicinal Chemistry Letters, 6:793-796; Diderichsen et al., 1996, Tett. Lett. 37:475-478; Fujii et al., 1997, Bioorg. Med. Chem. Lett. 7:637-627; Jordan et al., 1997, Bioorg. Med. Chem. Lett. 7:687-690; Krotz et al., 1995, Tett. Lett. 36:6941-6944; Lagriffoul et al., 1994, Bioorg. Med. Chem. Lett. 4:1081-1082; Diederichsen, 1997, Bioorg. Med. Chem. 25 Letters, 7:1743-1746; Lowe et al., 1997, J. Chem. Soc. Perkin Trans. 1, 1:539-546; Lowe et al., 1997, J. Chem. Soc. Perkin Trans. 11:547-554; Lowe et al., 1997, 1. Chem. Soc. Perkin Trans. 1 1:555-560; Howarth et al., 1997, I. Org. Chem. 62:5441-5450; Altmann et al., 1997, Bioorg. Med. Chem. Lett., 7:1119-1122; Diederichsen, 1998, Bioorg. Med. Chem. Lett., 8:165-168; Diederichsen et al., 1998, Angew. Chem. mt. Ed., 37:302-305; Cantin et al., 1997, Tett. Lett., 38:4211-4214; Ciapetti et al., 1997, Tetrahedron, 53:1167-1176; Lagriffoule et al., 1997, Chem. Eur. 1.′ 3:912-919; Kumar et al., 2001, Organic Letters 3(9):1269-1272; and the Peptide-Based Nucleic Acid Mimics (PENAMs) of Shah et al. as disclosed in WO 96/04000.

Some examples of PNAs are those in which the nucleobases are attached to an N-(2-aminoethyl)-glycine backbone, i.e., a peptide-like, amide-linked unit (see, e.g., U.S. Pat. No. 5,719,262; Buchardt et al., 1992, WO 92/20702; Nielsen et al., 1991, Science 254:1497-1500).

In some embodiments, a nucleobase polymer is a chimeric polymer. By “chimeric polymer” is meant a nucleobase polymer or oligomer comprising a plurality of different polynucleotides, polynucleotide analogs and polynucleotide mimics. For example a chimeric polymer may comprise a sequence of DNA linked to a sequence of RNA. Other examples of chimeric oligonucleotides include a sequence of DNA linked to a sequence of PNA, and a sequence of RNA linked to a sequence of PNA.

In some embodiments, a polynucleotide comprises one or more non-nucleobase moieties. Non-limiting examples of non-nucleobase moieties include but are not limited to a ligand, as described above, a “blocking moiety” suitable for inhibiting polymerase extension of the 3′ terminus of a flap probe when it is hybridized to a target sequence, and moieties suitable for producing a detectable signal. “Detectable moiety,” “detection moiety” or “label” refer to a moiety that, when attached to the disclosed polynucleotides and other compositions, render such compositions detectable or identifiable using known detection systems (e.g., spectroscopic, radioactive, enzymatic, chemical, photochemical, biochemical, immunochemical, chromatographic or electrophoretic systems). Non-limiting examples of labels include isotopic labels (e.g., radioactive or heavy isotopes), magnetic labels; spin labels, electric labels; thermal labels; colored labels (e.g., chromophores), luminescent labels (e.g., fluorescers, chemiluminescers), enzyme labels (e.g., horseradish peroxidase, alkaline phosphatase, luciferase, β-galactosidase) (Ichiki, et al.,1993, J. Immunol. 150(12):5408-5417; Nolan, et al., 1988, Proc. Natl. Acad. Sci. USA 85(8):2603-2607)), antibody labels, chemically modifiable labels, and mobility modifier labels. In addition, in some embodiments, such labels include components of ligand-binding partner pairs.

“Fluorescent label,” “fluorescent moiety,” and “fluorophore” refer to a molecule that may be detected via its inherent fluorescent properties. Examples of suitable fluorescent labels include, but are not limited to, fluorescein, rhodamine, tetramethylrhodamine, eosin, erythrosin, coumarin, methyl-coumarins, pyrene, Malacite Green, stilbene, Lucifer Yellow, Cascade BlueJ, Texas Red, IAEDANS, EDANS, BODIPY FL, LC Red 640, phycoerythrin, LC Red 705, Oregon green, Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE), FITC, Rhodamine, Texas Red (Pierce, Rockford, Ill.), Cy5, Cy5.5, Cy7 (Amersham Life Science, Pittsburgh, Pa.) and tandem conjugates, such as but not limited to, Cy5PE, Cy5.5PE, Cy7PE, Cy5.5APC, Cy7APC. In some embodiments, suitable fluorescent labels also include, but are not limited to, green fluorescent protein (GFP; Chalfie, et al., 1994, Science 263(5148):802-805), EGFP (Clontech Laboratories, Inc., Palo Alto, Calif.), blue fluorescent protein (BFP; Quantum Biotechnologies, Inc. Montreal, Canada; Heim et al, 1996, Curr. Biol. 6:178-182; Stauber, 1998, Biotechniques 24(3):462-471;), enhanced yellow fluorescent protein (EYFP; Clontech Laboratories, Inc., Palo Alto, Calif.), and renilla (WO 92/15673; WO 95/07463; WO 98/14605; WO 98/26277; WO 99/49019; U.S. Pat. Nos. 5,292,658, 5,418,155, 5,683,888, 5,741,668, 5,777,079, 5,804,387, 5,874,304, 5,876,995 and No. 5,925,558). Further examples of fluorescent labels are found in Haugland, Handbook of Fluorescent Probes and Research, 9^{th} Edition, Molecule Probes, Inc. Eugene, Oreg. (ISBN 0-9710636-0-5).

In other embodiments, a fluorescent moiety may be an acceptor or donor molecule of a fluorescence energy transfer (FET) or fluorescent resonance energy transfer (FRET) system, which utilize distance-dependent interactions between the excited states of two molecules in which excitation energy is transferred from a donor molecule to an acceptor molecule. (see Bustin., 2000, J. Mol. Endocrinol. 25:169-193; WO2004003510) As known in the art, these systems are suitable for detecting or monitoring changes in molecular proximity, including but not limited to, the release of the flap sequence by the 5′-3′ nuclease activity of a polymerase. Therefore, in some embodiments, a flap probe is labeled with donor and acceptor moieties, which provide a detection system suitable for monitoring the release of the flap sequence in real-time. In some embodiments, the transfer of energy from donor to acceptor results in the production of a detectable signal by the acceptor. In another embodiment, the transfer of energy from donor to acceptor results in quenching of the fluorescent signal produced by the donor. Thus, to detect or monitor the release of a flap sequence, the flap sequence and the target specific sequence each comprise a donor or acceptor moiety in energy transfer proximity. Therefore, depending upon the type of donor-acceptor moieties utilized, the release of the flap sequence may be detected or monitored by an increase or decrease in fluorescence signal. Examples of donor-acceptor pairs suitable for producing a fluorescent signal include but are not limited to fluorescein-tetramethylrhodamine, IAEDANS-fluorescein, EDANS-dabcyl, fluorescein-QSY 7, and fluorescein-QSY 9. Examples of donor-acceptor pairs suitable for quenching a fluorescent signal include but are not limited to FAM-DABCYL, HEX-DABCYL, TET-DABCYL, Cy3-DABCYL, Cy5-DABCYL, Cy5.5-DABCYL, rhodamine-DABCYL, TAMRA-DABCYL, JOE-DABCYL, ROX-DABCYL, Cascade Blue-DABCYL, Bodipy-DABCYL, FAM-MGB, Vic-MGB, Ned-MGB, ROX-MGB

In some embodiments, a label is an mobility modifier. “Mobility modifier” refers to a moiety capable of producing a particular mobility in a mobility-dependent analysis technique, such as, electrophoresis (see, e.g., U.S. Pat. Nos. 5,470,705, 5,514,543, 6,395,486 and 6,734,296). Thus, in some embodiments, a mobility modifier can be an electrophoresis mobility modifier, e.g., a nucleotide or a polynucleotide polymer (e.g., a ligation partner). In some embodiments, an electrophoresis mobility modifier is nonpolynucleotide polymer. Various non-limiting examples of non-polynucleotide electrophoresis mobility modifiers include but are not limited to polyethylene oxide, polyglycolic acid, polylactic acid, polypeptide, oligosaccharide, polyurethane, polyamide, polysulfonamide, polysulfoxide, polyphosphonate, and block copolymers thereof, including polymers composed of units of multiple subunits linked by charged or uncharged linking groups.

The use of detectable moieties in the detection or quantitation of target polynucleotides by the disclosed methods is within the abilities of the skilled artisan. Factors to be considered in selecting the number and types of detectable moieties and their distribution among the various polynucleotides, include but are not limited to, the number of target polynucleotides to be analyzed (e.g., single-plex vs. multiplex analysis), the method selected for detecting the released flap sequence, the number and types of detectable moieties than may be discriminated, and the extent to which each target sequence is to be discriminated. For example, in some embodiments, flap sequences may comprise detectable moieties. In some embodiments, such as multiplex target sequence analysis, each flap sequence may comprise a detectable moiety that may be discriminated from the detectable moieties of other flap sequences. Therefore, each released flap sequence may be identified by the emission of a unique signal. However, in some embodiments, each flap sequence may comprise the identical detectable moiety. In these embodiments, each released flap sequence may be individually discriminated if, for example, each flap sequence is substantially unique. For example, in embodiments in which each flap sequence differs in length by at least one nucleobase, the individual flap sequence may be conveniently discriminated by capillary electrophoresis. However, in embodiments in which each flap sequence comprises an identical detectable moiety and comprises a sequence of identical length, the individual flap sequences may be discriminated if, for example, the each flap sequence does not share statistically significant sequence homology with the other flap sequences. Therefore, in this embodiment, each released flap sequence may be ligated to a unique ligation partner each comprising a distinguishable mobility modifier, e.g., electrophoresis mobility modifier, to form distinguishable ligation amplicons, which also may be individually detected by capillary electrophoresis (e.g., ABI Prism® capillary electrophoresis instruments, Applied Biosystems, Foster City, Calif.).

Therefore, one advantage of the disclosed probes is their use in multiplex analysis of target polynucleotides. (see, e.g., U.S. Patent Application Ser. Nos. 60/584,596 and 60/584,643, each filed Jun. 30, 2004). Because the released flap sequences are an indicator of the presence of a target polynucleotide in a sample, the number of target polynucleotides that may be simultaneously analyzed is not limited by the number of detectable moieties that may be individually discriminated. For example, in some multiplex embodiments utilizing reporter molecules to analyze each target polynucleotide, each reporter molecule can produce a signal that is distinguishable from other reporter molecules. Therefore, the number of target polynucleotides analyzed in a multiplex format can be determined, at least in part, by the number of reporter molecules that may be discriminated. For example, in the embodiment, in which 5′ nuclease (e.g., TaqMan®) probes are utilized as reporter molecules about 2 to about 7 target sequences are analyzed in a multiplex reaction. However, in some embodiments in which flap probes are utilized as a reporter molecule about 2 to about 1,000 target sequences and in some embodiments to about 7,000 target sequences or more are analyzed in a multiplex reaction.

As will be appreciated by skilled artisans, target polynucleotides comprising one or more target sequences suitable analysis may be either DNA (e.g., cDNA, genomic DNA or extrachromosomal DNA) or RNA (e.g, mRNA, rRNA or genomic RNA) in nature, and may be derived or obtained from virtually any sample or source, wherein the sample may optionally be scarce or of a limited quantity. For example, the sample may be one or a few cells collected from a crime scene or a small amount of tissue collected via biopsy. In some embodiments, the target polynucleotide may be a synthetic polynucleotide comprising nucleotide analogs or mimics, as described above, produced for purposes, such as, diagnosis, testing, or treatment.

In various non-limiting examples, the target polynucleotide may be single or double-stranded or a combination thereof, linear or circular, a chromosome or a gene or a portion or fragment thereof, a regulatory polynucleotide, a restriction fragment from, for example, a plasmid or chromosomal DNA, genomic DNA, mitochondrial DNA, DNA from a construct or library of constructs (e.g., from a YAC, BAC or PAC library), RNA (e.g., mRNA, rRNA or vRNA) or a cDNA or a cDNA library.

In some embodiments, the target sequence may be amplified to produce a plurality of amplicons. As will be recognized by skilled artisans, methods for exponentially amplifying polynucleotide sequences are known in the art. For example, in some embodiments a target sequence may be amplified via the polymerase chain reaction (PCR; see, e.g., U.S. Pat. Nos. 4,683,195, 4,683,202, 4,800,159, 4,965,188, 5,075,216, 5,176,995, 5,386,022, 5,333,675, 5,656,493, 6,040,166, 6,197,563, 6,514,736, EP-A-0200362 and EP-A-0201184). In some embodiments, a target sequence is amplified via reverse transcription-PCR. Therefore, some embodiments include a reverse transcriptase and one or more primers suitable for reverse transcribing an RNA template into a cDNA. Reactions, reagents and conditions for carrying out such “RT” reactions are known in the art (see, e.g., Blain et al., 1993, J. Biol. Chem. 5:23585-23592; Blain et al., 1995, J. Virol. 69:4440-4452; PCR Essential Techniques 61-63, 80-81, (Burke, ed., J. Wiley & Sons 1996); Gübler et al., 1983, Gene 25:263-269; Gubler, 1987, Methods Enzymol., 152:330-335; Sellner et al., 1994, J. Virol. Method. 49:47-58; Okayama et al., 1982, Mol. Cell. Biol. 2:161-170; and U.S. Pat. Nos. 5,310,652, 5,322,770, and 6,300,073). In some embodiments, a target sequence may be exponentially amplified via the ligase chain reaction (LCR; see, e.g., U.S. Pat. Nos. 5,427,930, 5,516,663, 5,686,272, 5,869,252 and EP-A-320308. In some embodiments, a target sequence may be linearly amplified via polymerization reactions as described in, e.g., U.S. Pat. Nos. 5,066,584, 5,891,625 and WO 93/25706 or via a ligation reaction as described in, e.g., U.S. Pat. Nos. 5,185,243 and 5,679,524. In some embodiments, a target sequence may be amplified by a reaction that couples a linear amplification reaction to an exponential amplification reaction as described in U.S. Patent Application Ser. No. 60/584,665, filed Jun. 30, 2004. All of these various amplification methods can be utilized in various combinations to amplify target sequence either singly or in a multiplex format.

The target polynucleotide may include a single polynucleotide, from which one or more different target sequences of interest may be amplified, or it may include a plurality of different polynucleotides, from which one or more different target sequences of interest may be amplified. As will be recognized by skilled artisans, the sample or target polynucleotide may also include one or more polynucleotides comprising sequences that are not amplified in the log-linear reaction.

In some embodiments, highly complex mixtures of target sequences from highly complex mixtures of polynucleotides are amplified in either a single-plex or multiplex format. Indeed, many embodiments are suitable for multiplex analysis of target sequences from tens, hundreds, thousands, hundreds of thousands or even millions of polynucleotides. In some embodiments, multiplex amplification methods can be used to amplify pluralities of target sequences from samples comprising cDNA libraries or total mRNA isolated or derived from biological samples, such as tissues and/or cells, wherein the cDNA or, alternatively, mRNA libraries may be quite large. For example, cDNA libraries or mRNA libraries constructed from several organisms or from several different types of tissues or organs can be analyzed according to the methods described herein. As the skilled artisan will appreciate, multiple sets of primers and/or flap probes and/or polynucleotides suitable for detecting the released flap sequences are utilized for each target sequence to be analyzed by multiplex reactions.

The amount of target polynucleotide(s) included in the disclosed methods can vary widely. In many embodiments, amounts suitable for the amplification reactions, described above, may be used. For example, the target polynucleotide(s) may be from a single cell, from tens of cells, from hundreds of cells or even more, as is well known in the art. For many embodiments, including embodiments in which the target polynucleotide is a complex cDNA library (or derived therefrom by RT of mRNA), the total amount of target polynucleotide may range from about 1 pg to about 100 ng. For other embodiments, including embodiments in which the target polynucleotide(s) is obtained from a single cell, the total amount of target polynucleotide(s) may range from 1 copy (about 10 ag) to about 10⁷ copies (about 100 pg). In some embodiments, amounts of target polynucleotides suitable for detection or quantitation by the disclosed methods may be greater than about 100 ng, greater than about 500 ng, greater than about 1 μg or even greater. The skilled artisan will appreciate that as the amount of target polynucleotide sequence increases the requirement for target sequence amplification decreases. Therefore, in various embodiments, the number of amplification cycles may decreases or amplification of the target sequence may be avoided.

In some embodiments, preparation of the target polynucleotide(s) may not be required. In some embodiments, the target polynucleotide(s) may be prepared for log-linear amplification using conventional sample preparation techniques suitable for the type of amplification reaction to be used. For example, target polynucleotides may be isolated from their source via differential extraction, chromatography, precipitation, electrophoresis, as is well-known in the art. Alternatively, the target sequence(s) may be amplified directly from samples, including but not limited to, cells or from lysates of tissues or cells comprising the target polynucleotide(s).

The number of target sequences that can be amplified by a log-linear amplification is influenced in large part by the number of different amplification primers used during the log-linear amplification and the number of different methods used to detect or discriminate the amplification products. In some embodiments, at least two amplification primers are used for log-linear amplification of a target sequence. In other embodiments, at least three amplification primers are used. By “primer” herein are meant a polynucleotide capable of hybridizing or annealing to a template polynucleotide to form a substrate for a polymerase (e.g., DNA-dependent DNA polymerases, RNA-dependent DNA polymerase (reverse transcriptase)). Therefore, in various embodiments, a primer can be an amplification primer and/or a reverse transcription primer. By “annealing” or “hybridizing” is meant base-pairing interactions of one nucleobase polymer with another that results in the formation of a double-stranded structure. In some embodiments, annealing occurs via Watson-Crick base-pairing interactions, but may be mediated by other hydrogen-bonding interactions, such as Hoogsteen base pairing. When a primer is hybridized to its template, a polymerase initiates synthesis of a nascent polynucleotide strand in a template directed manner at the 3′ terminus of the primer.

Also provided are kits for use in practicing the various embodiments of the disclosed methods. Therefore, in some embodiments kits include one or more sets of amplification primers and flap probes to detect or quantitate one or more target polynucleotides. In some embodiments, a kit may further comprise a polymerase having 5′-3′ nuclease activity suitable for cleaving the flap sequence of the probe. In some embodiments, one or more primers comprise sequences of diversity and may be suitable for further rounds of amplification and/or hybridization to one or more probes. In some embodiments, kits may comprise polynucleotides suitable for detecting or modifying the released flap sequences. Therefore, in some embodiments, a kit also may comprise one or more enzyme suitable for modifying a released flap sequences, as described above.

The following examples are offered by way of illustration and not by way of limitation. All literature and similar materials cited in this application, including but not limited to, patents, patent applications, articles, books, and treatises, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

### 6. EXAMPLES

### Example 1

Ligation of Ligation Partners to Polynucleotides in a Multiplex Reaction:

To determine the detection sensitivity of capillary electrophoresis (CE) for analysis of ligated flap sequences, various amounts of fluorescent synthetic polynucleotide probes (1 nM, 100 pM, 10 pM, 1 pM; SEQ ID NOS:61-72) having a structure similar to released flap sequences were ligated to ligation partners (SEQ ID NOS:49-60) by hybridization to ligation templates (SEQ ID NOS:37-48) in the presence of a thermostable ligase.

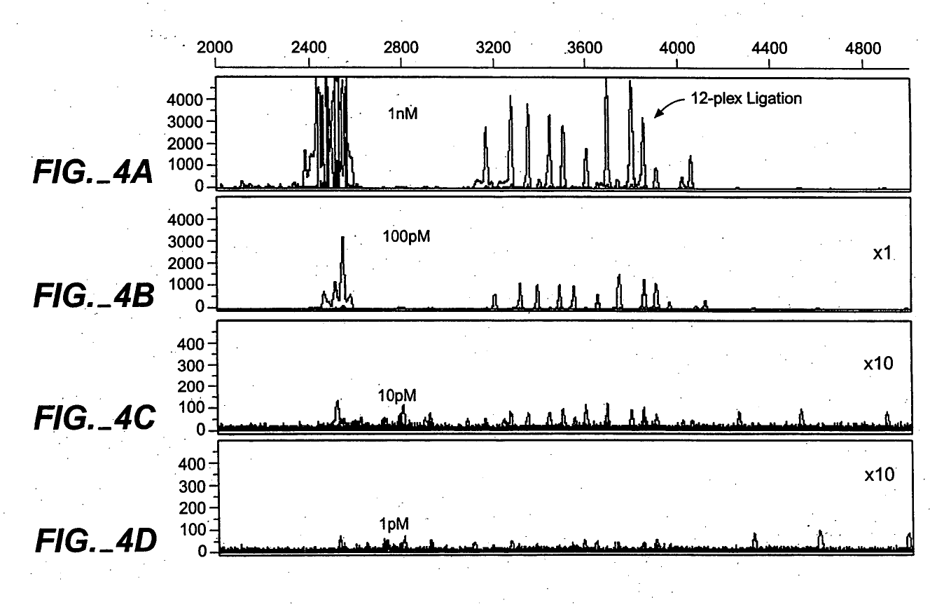

Each ligation reaction contained 1.0 μl 10× ABI Ligase Buffer (Applied Biosystems, Foster City, Calif.), 1 μl of a solution containing the 12 ligation sequences (100 nM), 1 μl of a solution containing the 12 ligation templates (100 nM), 5.75 μl ddH₂O, 0.25 μl AK16D ligase (Thernus sp. isolated AK16D; 40 U/μl) and 1.0 μl of a solution containing the synthetic probes (10 nM, 1000 pM, 100 pM or 10 pM). Each reaction was thermocycled 30 times (94° C. for 5 sec.-60° C. for 1 min.), and held at 4° C. For CE analysis 1.0 μl of each ligation reaction was added to 9.0 μl DI Formamide and injected onto an AB3100 capillary electrophoresis system (Applied Biosystems, Foster City, Calif.). FIG. 4A-D shows fluorescence intensity verses migration distance for the ligation amplicons. The amount of synthetic probe utilized in each reaction is shown in each panel. The results obtained with 10 pM and 1 pM synthetic probe were expanded 10-fold to improve visualization (10×).

### Example 2

Detection of Cox6B Sequence:

A 60-mer synthetic template (SEQ ID NO:74) having a 20 base pair portion of the Cox6b sequence flanked by primer sites was amplified by PCR and detected using a 40 base pair flap probe (SEQ ID NO:76) having a 5′ FAM fluorescent moiety.

Each PCR reaction contained 5 μl 2× ABI Master Mix (Applied Biosystems, Foster City, Calif.), 0.5 μl 10 μM forward primer (SEQ ID NO: 73), 0.5 μl 10 μM reverse primer (SEQ ID NO:75), 2.5 μl ddH₂O, 0.5 μl μM Coxb6 flap probe (SEQ ID NO:76), and 1.0 μl Cox6b template at a concentration of 1 pM, 100 fM or 10 fM. A negative control contained no template. The reaction was incubated at 95° C. for 10 min. and thermocycled 30 times (95° C. for 15 sec.-55° C. for 1 min.), incubated at 99° C. for 10 min. and held at 4° C.

The released flap sequences were ligated to a ligation partner (SEQ ID NO:60) by hybridization to a ligation template (SEQ ID NO:48) in the presence of a thermostable ligase and detected by CE. The ligation reaction contained 0.5 μl 10× ABI Ligase Buffer (Applied Biosystems, Foster City, Calif.), 1 μl ligation partner 100 nM (SEQ ID NO:60), 1.0 μl ligation template 100 nM (SEQ ID NO:60), 2 μl ddH₂O, 0.25 μl AK 16D Ligase (40 U/μl, Thermus sp. isolated AK16D) and 0.5 μl PCR reaction. The ligation reaction was thermocycled 30 times (94° C. for 5 sec.-60° C. for 1 min.). A 0.5 μl aliquot of the ligation reaction was mixed with 9.5 μl DI Formamide and analyzed by CE.

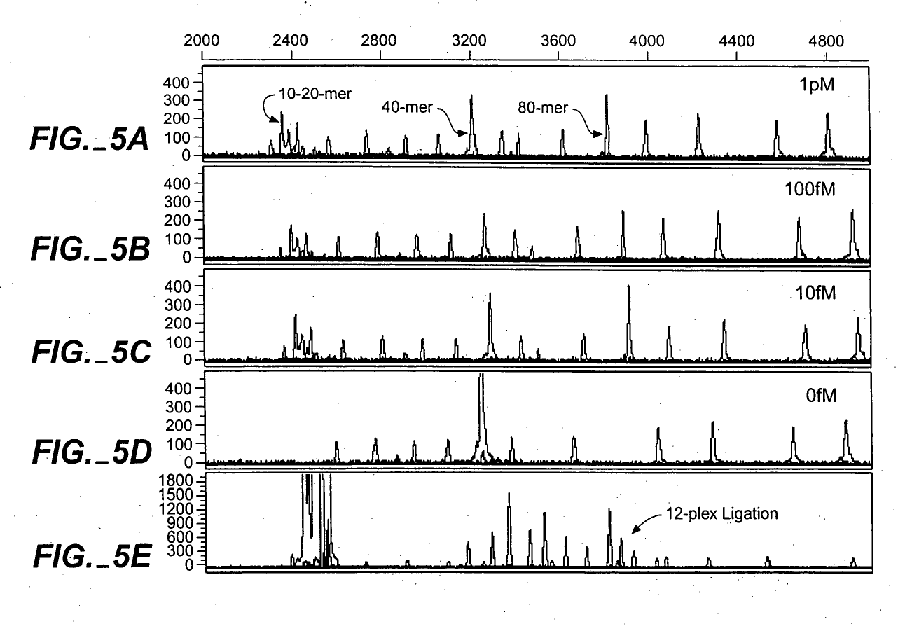

FIG. 5A shows the results obtained with 1 pM synthetic Cox6b template. The peaks labeled as 10⁻²⁰-mer are un-ligated released flap sequences. The peak labeled 40-mer is intact 40-mer flap probe. The peak labeled as about an 80-mer is the ligation amplicon of the released flap sequence ligated to the ligation partner. FIGS. 5B and 5C are the results obtained with 100 fM and 10 fM synthetic template. FIG. 5D shows the results of the negative control. FIG. 5E shows the results of the 12-plex ligation described in Example 1 that was included as a control. The peaks that are not labeled in FIG. 5 are internal molecule weight standards or markers that were co-electrophoresed.

TABLE 1

| | | |
|---|---|---|
| NAME | Flap Probes | SEQ ID NO: |

| | | |
|---|---|---|
| APOC2 | FAM-TGTGGGCGAAGCGGGAACCTCCTCCTCTCCCTTCAGC-MGB | SEQ ID NO:01 |
| ATP5B | FAM-TCGGAGCGATCACGTGGCACCATGGCTGAGACAAGAA-MGB | SEQ ID NO:02 |
| EIF1A | FAM-TACGCGACGCACCTGCTCCAACACCCGGCCCACGG-MGB | SEQ ID NO:03 |
| PPP1CA | FAM-TCCTCCCTCACGCGCCTGCAGAGGCACAAGTGGATGGT-MGB | SEQ ID NO:04 |
| PLAT | FAM-TGTGGACTGAGCGCGGATGGCCCAGGTCTGGGTGGTT-MGB | SEQ ID NO:05 |
| CETP | FAM-TCGAGGCAGACGCGTCCCACCAGCTGCTCTCAGTCAA-MGB | SEQ ID NO:06 |
| PEX7 | FAM-TTGGCCGAGACTGCAGGAGCGCCCGGTCACCAGCAA-MGB | SEQ ID NO:07 |
| ATP7A | FAM-TAGCGGACGACTGCGGACGAGAAACTGGCAGCACTCT-MGB | SEQ ID NO:08 |
| PRKCA | FAM-TGCCTGCGAAGACCCAAGCGACACAAGTTCAAAATC-MGB | SEQ ID NO:09 |
| BRCA1 | FAM-TGAGCAGCGACGCCGAGGCAGACCAGAGCTACAACAAT-MGB | SEQ ID NO:10 |
| MYL2 | FAM-TGCGAGTCCCGAGGGTCCCAATGGTCAGCATTTCC-MGB | SEQ ID NO:11 |
| Cox6b | FAM-TGGAAAGCGAGCGGCAGCCAAACACGCTGGTACCATT-MGB | SEQ ID NO:12 |

TABLE 2

| | | |
|---|---|---|
| NAME | Forward Primers | SEQ ID NO: |

| | | |
|---|---|---|
| APOC2 | CAGGCATTTTTACTGACCAAGTTCT | SEQ ID NO:13 |
| ATP5B | CCCGTGCACGGAAAATACAG | SEQ ID NO:14 |
| EIF1A | CAAGATTGGCGGCATTGG | SEQ ID NO:15 |
| PPP1CA | TTGTGCAGAAAAACAAGTCCTAAAGT | SEQ ID NO:16 |
| PLAT | CACATTGATGTCTCCTGCTGTACTAA | SEQ ID NO:17 |
| CETP | CAGATTACACCAAAGACTGTTTCCAA | SEQ ID NO:18 |
| PEX7 | TGAGTTGTGACTGGTGTAAATACAATGA | SEQ ID NO:19 |
| ATP7A | GGGAAGATGATGACACTGCATTATAA | SEQ ID NO:20 |
| PRKCA | ACTGATGACCCCAGGAGCAA | SEQ ID NO:21 |
| BRCA1 | CCAAAGACAGTCTTCTAATTCCTCATT | SEQ ID NO:22 |
| MYL2 | GGTGCTGAAGGCTGATTACGTT | SEQ ID NO:23 |
| Cox6b | ACCGCTAAAGGAGGCGATATC | SEQ ID NO:24 |

TABLE 3

| | | |
|---|---|---|
| NAME | Reverse Primers | SEQ ID NO: |

| | | |
|---|---|---|
| APOC2 | ACTCTCCCCTTGTCCACTGATG | SEQ ID NO:25 |
| ATP5B | CCTGTGAAGACCTCAGCAACCT | SEQ ID NO:26 |
| EIF1A | CCCGGCCGCAGGAT | SEQ ID NO:27 |
| PPP1CA | TGATTGGACATGACACAGGATACA | SEQ ID NO:28 |
| FLAT | AGCCCCACTGCGGTACTG | SEQ ID NO:29 |
| CETP | TGACTGCAGGAAGCTCTGGAT | SEQ ID NO:30 |
| PEX7 | AAGTCCCAGCCTCTCAAACTACAG | SEQ ID NO:31 |
| ATP7A | TGAATGCTGTGTCAGTGCATGA | SEQ ID NO:32 |
| PRKCA | GGTGGGGCTTCCGTAAGTGT | SEQ ID NO:33 |
| BRCA1 | TCATGCCAGAGGTCTTATATTTTAAGAG | SEQ ID NO:34 |
| MYL2 | CAACCTCCTCCTTGGAAAACC | SEQ ID NO:35 |
| Cox6b | TGGGGCAGAGGGACTGGTA | SEQ ID NO:36 |

TABLE 4

| | | DO NOT LIST |
|---|---|---|
| NAME | Probes | SEQ ID NO: |
| APOC2 | FAM-GTGGGCGAAGCGGGAACCTC | SEQ ID NO:61 |
| ATP5B | FAM-CGGAGCGATCACGTGGCACC | SEQ ID NO:62 |
| EIF1A | FAM-ACGCGACGCACCTGCTCCAA | SEQ ID NO:63 |
| PPP1CA | FAM-CCTCCCTCACGCGCCTGCAG | SEQ ID NO:64 |
| PLAT | FAM-GTGGACTGAGCGCGGATGGC | SEQ ID NO:65 |
| CETP | FAM-CGAGGCAGACGCGTCCCACC | SEQ ID NO:66 |
| PEX7 | FAM-TGGCCGAGACTGCAGGAGCG | SEQ ID NO:67 |
| ATP7A | FAM-AGCGGACGACTGCGGACGAG | SEQ ID NO:68 |
| PRKCA | FAM-GCCTGCGAAGACCCAAGCGA | SEQ ID NO:69 |
| BRCA1 | FAM-GAGCAGCGACGCCGAGGCAG | SEQ ID NO:70 |
| MYL2 | FAM-GCGAGTCCCGAGGGTCCCAA | SEQ ID NO:71 |
| Cox6b | FAM-GGAAAGCGAGCGGCAGCCAA | SEQ ID NO:72 |

TABLE 5

| | | |
|---|---|---|
| NAME | Ligation Templates | SEQ ID NO: |

| | | |
|---|---|---|
| APOC2 | TTGGCCTGTTCCCGCTCGCTGAGGTTCCCGCTTCGCCCAC-NH2 | SEQ ID NO:37 |
| ATP5B | CCTGCGCTGCGTGGTGTTCCGGTGCCACGTGATCGCTCCG-NH2 | SEQ ID NO:38 |
| EIF1A | TCGCGGACGGTGAGCACTGCTTGGAGCAGGTGCGTCGCGT-NH2 | SEQ ID NO:39 |
| PPP1CA | TTCCCGCTGGTGCCACTCCGCTGCAGGCGCGTGAGGGAGG-NH2 | SEQ ID NO:40 |
| PLAT | CGCTCGCTTTGGCCTGCTGCGCCATCCGCGCTCAGTCCAC-NH2 | SEQ ID NO:41 |
| CETP | CGCTGCGTGAGGGCTCGGACGGTGGGACGCGTCTGCCTCG-NH2 | SEQ ID NO:42 |
| PEX7 | TTCCACCCGCGTCGTCCTGCCGCTCCTGCAGTCTCGGCCA-NH2 | SEQ ID NO:43 |
| ATP7A | GACCGCTCAGCAGACCGCCACTCGTCCGCAGTCGTCCGCT-NH2 | SEQ ID NO:44 |
| PRKCA | AGCACGCTTCCGGGACCCACTCGCTTGGGTCTTCGCAGGC-NH2 | SEQ ID NO:45 |
| BRCA1 | AGGCGGTGTTGGCTCGAGGCCTGCCTCGGCGTCGCTGCTC-NH2 | SEQ ID NO:46 |
| MYL2 | GCGTGTCTCCTGTCCGGCCATTGGGACCCTCGGGACTCGC-NH2 | SEQ ID NO:47 |
| Cox6b | TTGGGCTCGTGAAGGCAGGCTTGGCTGCCGCTCGCTTTCC-NH2 | SEQ ID NO:48 |

TABLE 6

| | | |
|---|---|---|
| NAME | Ligation Partners | SEQ ID NO: |

| | | |
|---|---|---|
| APOC2 | PO₄-AGCGAGCGGGAACAGGCCAA(T)₂ | SEQ ID NO:49 |
| ATP5B | PO₄-GGAACACCACGCAGCGCAGG(T)₅ | SEQ ID NO:50 |
| EIF1A | PO₄-GCAGTGCTCACCGTCCGCGA(T)₈ | SEQ ID NO:51 |
| PPP1CA | PO₄-CGGAGTGGCACCAGCGGGAA(T)₁₁ | SEQ ID NO:52 |
| PLAT | PO₄-GCAGCAGGCCAAAGCGAGCG(T)₁₄ | SEQ ID NO:53 |
| CETP | PO₄-GTCCGAGCCCTCACGCAGCG(T)₁₇ | SEQ ID NO:54 |
| PEX7 | PO₄-GCAGGACGACGCGGGTGGAA(T)₂₀ | SEQ ID NO:55 |
| ATP7A | PO₄-TGGCGGTCTGCTGAGCGGTC(T)₂₃ | SEQ ID NO:56 |
| PRKCA | PO₄-GTGGGTCCCGGAAGCGTGCT(T)₂₆ | SEQ ID NO:57 |
| BRCA1 | PO₄-GCCTCGAGCCAACACCGCCT(T)₂₉ | SEQ ID NO:58 |
| MYL2 | PO₄-TGGCCGGACAGGAGACACGC(T)₃₂ | SEQ ID NO:59 |
| Cox6b | PO₄-GCCTGCCTTCACGAGCCCAA(T)₃₅ | SEQ ID NO:60 |

What is claimed is:

1. A method of analyzing a target sequence comprising:
a) contacting a target sequence with a polymerase, a primer, and a probe, wherein said polymerase comprises 5′-3′ nuclease activity, said primer comprises a sequence suitable for amplifying said target sequence, and said probe comprises a 5′ sequence and a 3′ sequence, wherein said 5′ sequence is not suitable for hybridizing to said target sequence and said 3′ sequence is suitable for hybridizing to said target sequence 3′ relative to said primer, under conditions suitable for said primer and said probe to hybridize to said target sequence to form a substrate for said nuclease activity and for said nuclease activity to release said 5′ sequence from said probe;
b) ligating the released 5′ sequence to a ligation partner to form a ligation product, wherein said ligation product is formed by hybridizing said released sequence and said ligation partner to a template under conditions effective for a ligase to form a bond between said released sequence and said ligation partner; and
c) detecting said ligation product.

2. The method according to claim 1, wherein said 5′ sequence comprises a first detection moiety.

3. The method according to claim 2, wherein said first detection moiety is a fluorophore.

4. The method according to claim 2, wherein said first detection moiety is an mobility modifier.

5. The method according to claim 1, wherein said ligation partner comprises a second detection moiety.

6. The method according to claim 5, wherein said second detection moiety is a fluorophore.

7. The method according to claim 5, wherein said second detection moiety is an mobility modifier.

8. The method according to claim 1, wherein said ligation product is detected by capillary electrophoresis.

9. The method according to claim 1, wherein the 3′ terminus of said primer hybridized to said target sequence is within about 20 nucleotides of the 5′ nucleotide of said probe hybridized to said target sequence, thereby having spacing effective to permit the release of said 5′ sequence in the absence of nucleic acid polymerization.

10. The method according to claim 1, wherein said polymerase is a thermostable polymerase.

11. The method according to claim 1, wherein said target sequence is RNA and step “a” is further carried out in the presence of reverse transcriptase and a primer suitable to generate a cDNA of said RNA.

12. The method according to claim 1, wherein the 3′ terminus of said probe is not a suitable substrate to prime nucleic acid synthesis.

13. The method according to claim 1, wherein said probe comprises a labeling system suitable for monitoring the release of said 5′ sequence as a function of time and said method further comprises monitoring the release of said 5′ sequence.

14. A method of analyzing a target sequence, comprising:
a) amplifying a target sequence with a polymerase having 5′-3′ nuclease activity, a pair of amplification primers, and a probe, wherein said probe comprises a 5′ sequence and a 3′ sequence, and wherein said 5′ sequence is not suitable for hybridizing to said target sequence and comprises a fluorophore, and said 3′ sequence is suitable for hybridizing to said target sequence 3′ relative to said primer, wherein the amplification conditions are effective to hybridize said primer and said probe to said target sequence to form a substrate for said nuclease activity and for said nuclease activity to release said 5′ sequence from said probe;
b) ligating the released 5′ sequence to a ligation partner having an electrophoresis mobility modifier, to form a ligation product, wherein said ligation product is formed by hybridizing said released sequence and said ligation partner to a template under conditions effective for a ligase to form a bond between said released sequence and said ligation partner; and
c) detecting said ligation product by capillary electrophoresis.

15. The method according to claim 14, wherein said polymerase is a thermostable polymerase.

16. The method according to claim 14, wherein said target sequence is RNA and step “a” is further carried out in the presence of reverse transcriptase and a primer suitable to generate a cDNA of said RNA.

17. The method according to claim 14, wherein the 3′ terminus of said probe is not a suitable substrate to prime nucleic acid synthesis.

18. The method according to claim 14, wherein said probe comprises a labeling system suitable for monitoring the release of said 5′ sequence as a function of time and said method further comprises monitoring the release of said 5′ sequence.

19. A method of analyzing a plurality of target sequences in a multiplex format, comprising:
a) contacting a plurality of target sequences with a polymerase, a plurality of primers, and a plurality of probes, wherein said polymerase comprises 5′-3′ nuclease activity, said primers comprise sequences suitable for amplifying said target sequences, and said probes comprise 5′ sequences and 3′ sequences, wherein said 5′ sequences are not suitable for hybridizing to said target sequences and comprise distinguishable nucleotide sequences, and said 3′ sequences are suitable to hybridizing to said target sequences 3′ relative to said primers, and wherein the conditions are suitable for said primers and said probes to hybridize to said target sequences to form a plurality of substrates for said nuclease activity and for said nuclease activity to release said ₅′ sequences from said probes;
b) ligating the released 5′ sequences to a plurality of ligation partners to form a plurality of ligation products, wherein each ligation partner comprises a distinguishable nucleotide sequence, and said ligation products are formed by hybridizing said released sequences and said ligation partners to a plurality of distinguishable templates under conditions effective for a ligase to form a bond between said released and said ligation partners; and
c) detecting said ligation products.

20. The method according to claim 19, wherein said 5′ sequences comprise a fluorophore.

21. The method according to claim 19, wherein said 5′ sequences each comprise a distinguishable mobility modifier.

22. The method according to claim 19, wherein said ligation partners comprise a fluorophore.

23. The method according to claim 19, wherein said ligation partners each comprise a distinguishable mobility modifier.

24. The method according to claim 19, wherein said ligation products are detected by capillary electrophoresis.

25. The method according to claim 19, wherein the 3′ termini of said primers hybridized to said target sequences are within about 20 nucleotides of the 5′ nucleotides of said probes hybridized to said target sequences, thereby having spacing effective to permit the release of said 5′ sequences in the absence of nucleic acid polymerization.

26. The method according to claim 19, wherein said polymerase is a thermostable polymerase.

27. The method according to claim 19, wherein said target sequences are RNA and step “a” is further carried out in the presence of reverse transcriptase and a plurality of primers suitable to generate cDNA of said RNA.

28. The method according to claim 19, wherein the 3′ termini of said probes are not suitable substrates for said polymerase.

29. The method according to claim 19, wherein said plurality of probes each comprise a distinguishable labeling system suitable for monitoring the release of said 5′ sequences as a function of time and wherein said method further comprises monitoring the release of said 5′ sequences.

30. A method of analyzing a plurality of target sequences in a multiplex format, comprising:
a) amplifying a plurality of target sequences with a polymerase having 5′-3′ nuclease activity and a plurality of amplification primer pairs in the presence of a plurality of probes, wherein said probes comprise 5′ sequences and 3′ sequences, wherein said 5′ sequences are not suitable for hybridizing to said target sequences, comprise a fluorophore, and comprise distinguishable nucleotide sequences, and said 3′ sequences are suitable for hybridizing to said target sequences, and wherein the conditions are suitable for said primers and said probes to hybridize to said target sequences to form a plurality of substrates for said nuclease activity and for said nuclease activity to release said 5′ sequences from said probes;
b) ligating the released 5′ sequences to a plurality of ligation partners to form a plurality of ligation products, wherein each ligation partner comprises a distinguishable nucleotide sequence and electrophoresis mobility modifier, and said ligation products are formed by hybridizing said released sequences and said ligation partners to a plurality of distinguishable templates under conditions effective for a ligase to form a bond between said released sequences and said ligation partners; and
c) detecting said ligation products by capillary electrophoresis.

31. The method according to claim 30, wherein said polymerase is a thermostable polymerase.

32. The method according to claim 30, wherein said target sequences are RNA and step “a” is further carried out in the presence of reverse transcriptase and a plurality of primers suitable to generate cDNA of said RNA.

33. The method according to claim 30, wherein said probe comprises a 3′ terminus that it not a suitable substrate for said polymerase.

34. The method according to claim 30, wherein said plurality of probes each comprise a distinguishable labeling system suitable for monitoring the release of said 5′ sequences as a function of time and wherein said method further comprises monitoring the released 5′ sequences.

35. A kit for detecting of a target sequence comprising, in one or more reaction vessels:
a) a pair of primers suitable for amplifying a target sequence;
b) a probe comprising a 5′ sequence and a 3′ sequence, wherein said 5′ sequence is not suitable for hybridizing to said target sequence and comprises a fluorophore, and said 3′ sequence is suitable for hybridizing to said target sequence, wherein said primers, said probe, and said target sequence, under hybridization conditions, form a substrate for the 5′-3′ nuclease activity of a polymerase to release said 5′ sequence from said probe;
c) a ligation partner comprising an mobility modifier, and
d) a template comprising sequences suitable for hybridizing to said ligation partner and the released 5′ sequence to form a substrate for a ligase.

36. A kit for detecting of a plurality of target sequences comprising, in one or more reaction vessels:
a) a plurality of primer pairs suitable for amplifying a plurality of target sequences;
b) a plurality of probes comprising 5′ sequences and 3′ sequences, wherein said 5′ sequences are not suitable for hybridizing to said target sequences, comprise a fluorophore, and each comprise a distinguishable nucleotide sequence, and wherein said 3′ sequences are suitable for hybridizing to said target sequences, wherein said primers, said probes, and said target sequences, under hybridizing conditions, form a plurality of substrates for the 5′-3′ nuclease activity of a polymerase to release said 5′ sequences from said probes;
c) a plurality of ligation partners each comprising a distinguishable nucleotide sequence and mobility modifier, and
d) a plurality of templates comprising sequences suitable for hybridizing to said ligation partners and the released 5′ sequences to form substrates for a ligase to join each of the released 5′ sequences to one of the plurality of ligation partners.
